(19) United States
(12) Patent Application Publication
(10) Pub. No.: US 2011/0070190 A1
(43) Pub. Date: Mar. 24, 2011

(54) HETEROCYCLIC ANTIVIRAL COMPOUNDS

(75) Inventors: Chris Allen Broka, Las Vegas, NV (US); Robert Than Hendricks, San Carlos, CA (US); Hans Maag, Sausalito, CA (US); David Bernard Smith, San Mateo, CA (US); Jutta Wanner, Montclair, NJ (US)

(73) Assignee: Roche Palo Alto LLC, Palo Alto, CA (US)

(21) Appl. No.: 12/890,218

(22) Filed: Sep. 24, 2010

Related U.S. Application Data
(60) Provisional application No. 61/245,497, filed on Sep. 24, 2009.

(51) Int. Cl. A61K 31/4365; C07D 495/04; A61K 31/437; A61K 39/00; A61K 38/21; A61K 38/20; A61K 38/19; A61P 31/14; C12N 5/02

(52) U.S. Cl. 424/85.2; 544/278; 544/281; 514/301; 424/184.1; 424/85.4; 424/85.1; 435/375

(57) ABSTRACT

The invention discloses 1-N-substituted-6-(hetero)aryl-1H-thieno[3,2-d]pyrimidin-4-one derivatives of formula

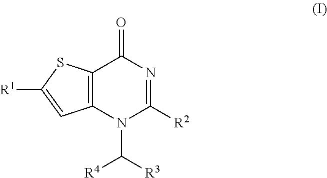

wherein R¹, R², R³ and R⁴ are as defined herein that inhibit Hepatitis C virus NS5b polymerase inhibitors. Also disclosed are compositions and methods for treating an HCV infection and inhibiting HCV replication.

### CROSS-REFERENCE TO PRIOR APPLICATIONS

This application claims the benefit of priority to U.S. Ser. No. 61/245,497 filed Sep. 24, 2009 which is hereby incorporated by reference in their entirety.

### FIELD OF THE INVENTION

The present invention provides non-nucleoside compounds of formula I, and certain derivatives thereof, which are inhibitors of RNA-dependent RNA viral polymerase. These compounds are useful for the treatment of RNA-dependent RNA viral infection. They are particularly useful as inhibitors of hepatitis C virus (HCV) NS5B polymerase, as inhibitors of HCV replication, and for the treatment of hepatitis C infection.

### BACKGROUND

Hepatitis C virus is the leading cause of chronic liver disease throughout the world. (Boyer, N. et al., J. Hepatol. 2000 32:98-112). Patients infected with HCV are at risk of developing cirrhosis of the liver and subsequent hepatocellular carcinoma and hence HCV is the major indication for liver transplantation.

HCV has been classified as a member of the virus family Flaviviridae that includes the genera flaviviruses, pestiviruses, and hapaceiviruses which includes hepatitis C viruses (Rice, C. M., Flaviviridae: The viruses and their replication. In: Fields Virology, Editors: B. N. Fields, D. M. Knipe and P. M. Howley, Lippincott-Raven Publishers, Philadelphia, Pa., Chapter 30, 931-959, 1996). HCV is an enveloped virus containing a positive-sense single-stranded RNA genome of approximately 9.4 kb. The viral genome consists of a highly conserved 5′ untranslated region (UTR), a long open reading frame encoding a polyprotein precursor of approximately 3011 amino acids, and a short 3′ UTR.

Genetic analysis of HCV has identified six main genotypes which diverge by over 30% of the DNA sequence. More than 30 subtypes have been distinguished. In the US approximately 70% of infected individuals have Type 1a and 1b infection. Type 1b is the most prevalent subtype in Asia. (X. Forms and J. Bukh, Clinics in Liver Disease 1999 3:693-716; J. Bukh et al., Semin. Liv. Dis. 1995 15:41-63). Unfortunately Type 1 infectious is more resistant to therapy than either type 2 or 3 genotypes (N. N. Zein, Clin. Microbiol. Rev., 2000 13:223-235).

Viral structural proteins include a nucleocapsid core protein (C) and two envelope glycoproteins, E1 and E2. HCV also encodes two proteases, a zinc-dependent metalloproteinase encoded by the NS2-NS3 region and a serine protease encoded in the NS3 region. These proteases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown. It is believed that most of the non-structural proteins encoded by the HCV RNA genome are involved in RNA replication

Currently a limited number of approved therapies are available for the treatment of HCV infection. New and existing therapeutic approaches for treating HCV infection and inhibiting of HCV NS5B polymerase activity have been reviewed: R. G. Gish, Sem. Liver. Dis., 1999 19:5; Di Besceglie, A. M. and Bacon, B. R., Scientific American, October: 1999 80-85; G. Lake-Bakaar, Current and Future Therapy for Chronic Hepatitis C Virus Liver Disease, Curr. Drug Targ. Infect Dis. 2003 3(3):247-253; P. Hoffmann et al., Recent patent on experimental therapy for hepatitis C virus infection (1999-2002), Exp. Opin. Ther. Patents 2003 13(11):1707-1723; M. P. Walker et al., Promising Candidates for the treatment of chronic hepatitis C, Exp. Opin. Investing. Drugs 2003 12(8):1269-1280; S.-L. Tan et al., Hepatitis C Therapeutics: Current Status and Emerging Strategies, Nature Rev. Drug Discov. 2002 1:867-881; J. Z. Wu and Z. Hong, Targeting NS5B RNA-Dependent RNA Polymerase for Anti-HCV Chemotherapy, Curr. Drug Targ.-Infect. Dis. 2003 3(3):207-219.

Ribavirin (1-((2R,3R,4S,5R)-3,4-Dihydroxy-5-hydroxymethyl-tetrahydro-furan-2-yl)-1H-[1,2,4]triazole-3-carboxylic acid amide; Virazole®) is a synthetic, non-interferon-inducing, broad-spectrum antiviral nucleoside analog. Ribavirin has in vitro activity against several DNA and RNA viruses including Flaviviridae (Gary L. Davis. Gastroenterology 2000 118:S104-S114). Although, in monotherapy ribavirin reduces serum amino transferase levels to normal in 40% of patients, it does not lower serum levels of HCV-RNA. Ribavirin also exhibits significant toxicity and is known to induce anemia. Viramidine is a ribavirin prodrug converted ribavirin by adenosine deaminase to in hepatocytes. (J. Z. Wu, Antivir. Chem. Chemother. 2006 17(1):33-9)

Interferons (IFNs) have been available for the treatment of chronic hepatitis for nearly a decade. IFNs are glycoproteins produced by immune cells in response to viral infection. Two distinct types of interferon are recognized: Type 1 includes several interferon alphas and one interferon beta, type 2 includes interferon gamma. Type 1 interferons are produced mainly by infected cells and protect neighboring cells from de novo infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary. Cessation of therapy results in a 70% relapse rate and only 10-15% exhibit a sustained virological response with normal serum alanine transferase levels. (Davis, Luke-Bakaar, supra)

One limitation of early IFN therapy was rapid clearance of the protein from the blood. Chemical derivatization of IFN with polyethyleneglycol (PEG) has resulted in proteins with substantially improved pharmacokinetic properties. PEGASYS® is a conjugate interferon α-2a and a 40 kD branched mono-methoxy PEG and PEG-INTRONO is a conjugate of interferon α-2b and a 12 kD mono-methoxy PEG. (B. A. Luxon et al., Clin. Therap. 2002 24(9):13631383; A. Kozlowski and J. M. Harris, J. Control. Release 2001 72:217-224).

Combination therapy of HCV with ribavirin and interferon-α currently is the optimal therapy for HCV. Combining ribavirin and PEG-IFN (infra) results in a sustained viral response (SVR) in 54-56% of patients with type 1 HCV. The SVR approaches 80% for type 2 and 3 HCV. (Walker, supra) Unfortunately, combination therapy also produces side effects which pose clinical challenges. Depression, flu-like symptoms and skin reactions are associated with subcutaneous IFN-α and hemolytic anemia is associated with sustained treatment with ribavirin.

A number of potential molecular targets for drug development as anti-HCV therapeutics have now been identified including, but not limited to, the NS2-NS3 autoprotease, the NS3 protease, the NS3 helicase and the NS5B polymerase. The RNA-dependent RNA polymerase is absolutely essential for replication of the single-stranded, positive sense, RNA genome. This enzyme has elicited significant interest among medicinal chemists.

Nucleoside inhibitors can act either as a chain terminator or as a competitive inhibitor that interferes with nucleotide binding to the polymerase. To function as a chain terminator the nucleoside analog must be taken up by the cell in vivo and be converted in vivo to its triphosphate form to compete as a substrate at the polymerase nucleotide binding site. This conversion to the triphosphate is commonly mediated by cellular kinases which impart additional structural limitations on any nucleoside. In addition this requirement for phosphorylation limits the direct evaluation of nucleosides as inhibitors of HCV replication to cell-based assays (J. A. Martin et al., U.S. Pat. No. 6,846,810; C. Pierra et al., J. Med. Chem. 2006 49(22):6614-6620; J. W. Tomassini et al., Antimicrob. Agents and Chemother. 2005 49(5):2050; J. L. Clark et al., J. Med. Chem. 2005 48(17):2005).

Compounds of the present invention and their isomeric forms and pharmaceutically acceptable salts thereof are also useful in treating viral infections, in particular, hepatitis C infection, and diseases in living hosts when used in combination with each other and with other biologically active agents, including but not limited to the group consisting of interferon, a pegylated interferon, ribavirin, protease inhibitors, polymerase inhibitors, small interfering RNA compounds, antisense compounds, nucleotide analogs, nucleoside analogs, immunoglobulins, immunomodulators, hepatoprotectants, anti-inflammatory agents, antibiotics, antivirals and antiinfective compounds. Such combination therapy may also comprise providing a compound of the invention either concurrently or sequentially with other medicinal agents or potentiators, such as ribavirin and related compounds, amantadine and related compounds, various interferons such as, for example, interferon-alpha, interferon-beta, interferon gamma and the like, as well as alternate forms of interferons such as pegylated interferons. Additionally combinations of ribavirin and interferon, may be administered as an additional combination therapy with at least one of the compounds of the present invention.

Other interferons currently in development include albinterferon-α-2b (Albuferon), IFN-omega with DUROS, LOCTERON™ and interferon-α-2b XL. As these and other interferons reach the marketplace their use in combination therapy with compounds of the present invention is anticipated.

HCV polymerase inhibitors are another target for drug discovery and compounds in development include R-1626, R-7128, IDX184/IDX102, PF-868554 (Pfizer), VCH-759 (ViroChem), GS-9190 (Gilead), A-837093 and A-848837 (Abbot), MK-3281 (Merck), GSK949614 and GSK625433 (Glaxo), ANA598 (Anadys), VBY 708 (ViroBay).

Inhibitors of the HCV NS3 protease also have been identified as potentially useful for treatment of HCV. Protease inhibitors in clinical trials include VX-950 (Telaprevir, Vertex), SCH503034 (Broceprevir, Schering), TMC435350 (Tibotec/Medivir) and ITMN-191 (Intermune). Other protease inhibitors in earlier stages of development include MK7009 (Merck), BMS-790052 (Bristol Myers Squibb), VBY-376 (Virobay), IDXSCA/IDXSCB (Idenix), BI12202 (Boehringer), VX-500 (Vertex), PHX1766 Phenomix).

Other targets for anti-HCV therapy under investigation include cyclophilin inhibitors which inhibit RNA binding to NS5b, nitazoxanide, Celgosivir (Migenix), an inhibitor of α-glucosidase-1, caspase inhibitors, Toll-like receptor agonists and immunostimulants such as Zadaxin (SciClone).

### SUMMARY OF THE INVENTION

There is currently no preventive treatment of Hepatitis C virus (HCV) and currently approved therapies, which exist only against HCV, are limited. Design and development of new pharmaceutical compounds is essential.

The present invention provides a compound according to formula I, or a pharmaceutically acceptable salt thereof, wherein:

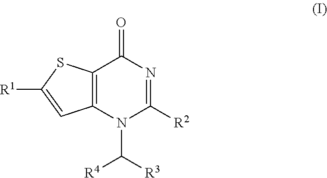

R¹ is phenyl or pyridinyl optionally substituted with one to three groups selected from the group consisting of:
(a) C₁₋₆ alkyl,
(b) C₁₋₆ alkoxy,
(c) halogen,
(d) phenyl-C₁₋₆ alkoxy said phenyl optionally independently substituted by one to three groups selected from C₁₋₃ alkoxy, halogen or C₁₋₃ alkyl or C₁₋₃-haloalkyl,
(e) phenyl,
(f) heteroaryl-C₁₋₃ alkoxy wherein the heteroaryl group is pyridinyl, pyrimidinyl or pyrazinyl said heteroaryl optionally independently substituted by one or two groups selected from amino, C₁₋₆ alkyl, halogen or C₁₋₆ alkoxy;
(g) phenoxymethyl optionally independently substituted by one or two groups selected from amino, C₁₋₆ alkyl, halogen or C₁₋₆ alkoxy;
(h) pyridinylmethylsulfanyl,
(i) heteroaryl wherein the heteroaryl group is pyridinyl, [1,3,4]oxadiazol-2-yl, furo[3,2-b]pyridine-2-yl, pyrazolo[1,5-a]pyrimidin-2-yl and said heteroaryl is optionally independently substituted by one to three groups selected from C₁₋₆ alkyl, C₁₋₆ alkoxy, halogen, amino, C₁₋₃ alkylamino, C₁₋₃ dialkylamino, a cyclic amine,
(j) phenyl-C₁₋₃ alkylsulfanyl,
(k) hydroxy,
(l) halogen,
(m) carboxyl,
(n) cyano,
(p) C₁₋₆ hydroxyalkyl,
(p) CONR^{c}R^{d},
(q) NR^{a}R^{b},
(r) NHC(O)NR^{g}R^{h}, and
(s) hydrogen.

R² is halogen, C₁₋₃ alkyl or C₁₋₃ alkoxy and n is 0 to 2.

R^{a} and R^{b} (i) taken individually are independently:
(a) C₁₋₆ alkoxycarbonyl,
(b) benzyl,
(c) hydroxy-C₁₋₆ alkanoyl,
(d) acyl,
(e) phenylcarbonyl said phenyl optionally independently substituted with one to three groups selected from C₁₋₃ alkoxy, halo, hydroxy,
(f) heteroarylcarbonyl wherein said heteroaryl group is optionally substituted pyrazole, 2-methyl-furan-5-yl-carbonyl, pyrimidinyl-4-carbonyl, oxazol-5-yl-carbonyl, pyrazin-2-yl-carbonyl, pyridinyl-carbonyl said heteroarylcarbonyl optionally substituted by one or two groups independently selected from C₁₋₆ alkyl, C₁₋₆ alkoxy, halogen, amino, C₁₋₃ alkylamino, C₁₋₃ dialkylamino, a cyclic amine or C₁₋₆ hydroxyalkoxy,
(g) hydrogen, or
(ii) taken together with the nitrogen to which they are attached are a cyclic amine.

R^{c} and R^{d} are independently hydrogen, C₁₋₆ alkyl or phenyl.

R³ is phenyl optionally substituted with one to three groups selected from the group consisting of (a) C₁₋₆ alkyl, (b) C₁₋₆ alkoxy, (c) halogen, (d) NR^{e}R^{f}, (e) cyano, (f) C₁₋₃ haloalkyl and (g) hydroxy, or, C₃₋₇ cycloalkyl optionally with one to three groups selected from C₁₋₄ alkyl, halogen or C₁₋₄ alkoxy.

R^{e} and R^{f} are independently hydrogen, C₁₋₆ alkyl, C₁₋₆ sulfonyl.

R^{g} and R^{h} are independently hydrogen or C₁₋₃ alkyl or together with the nitrogen to which they are attached form a pyrrolidine or a piperidine.

R⁴ is hydrogen or C₁₋₆ alkyl.

Compounds of general formula I can be either neutral compounds or a pharmaceutically acceptable salt thereof.

The present invention also provides a method for treating a disease a Hepatitis C Virus (HCV) virus infection by administering a therapeutically effective quantity of a compound according to formula I to a patient in need thereof. The compound can be administered alone or co-administered with other antiviral compounds or immunomodulators.

The present invention also provides a method for inhibiting replication of HCV in a cell by administering a compound according to formula I in an amount effective to inhibit HCV.

The present invention also provides a pharmaceutical composition comprising a compound according to formula I and at least one pharmaceutically acceptable carrier, diluent or excipient.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:

MeC(═O)OR⁴ wherein R⁴=

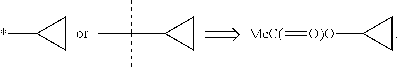

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

It will be appreciated by the skilled artisan that some of the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g. enantiomers), racemic mixtures or partially resolved mixtures of the compounds of formulae I and, where appropriate, the individual tautomeric forms thereof.

The racemates can be used as such or can be resolved into their individual isomers. The resolution can afford stereo chemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively individual isomers can be separated chemically from a mixture by forming diastereomeric salts with a chiral acid, such as the individual enantiomers of 10-camphorsulfonic acid, camphoric acid, .alpha.-bromocamphoric acid, tartaric acid, diacetyltartaric acid, malic acid, pyrrolidone-5-carboxylic acid, and the like, fractionally crystallizing the salts, and then freeing one or both of the resolved bases, optionally repeating the process, so as obtain either or both substantially free of the other; i.e., in a form having an optical purity of >95%. Alternatively the racemates can be covalently linked to a chiral compound (auxiliary) to produce diastereomers which can be separated by chromatography or by fractional crystallization after which time the chiral auxiliary is chemically removed to afford the pure enantiomers.

The compounds of formula I may contain a basic center and suitable acid addition salts are formed from acids which form non-toxic salts. Examples of salts of inorganic acids include the hydrochloride, hydrobromide, hydroiodide, chloride, bromide, iodide, sulfate, bisulfate, nitrate, phosphate, hydrogen phosphate. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulfate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts see Berge et al, J. Pharm. Sci., 1977 66:1-19 and G. S. Paulekuhn et al. J. Med. Chem. 2007 50:6665.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001). The starting materials and reagents used in preparing these compounds generally are either available from commercial suppliers, such as Aldrich Chemical Co., or are prepared by methods known to those skilled in the art following procedures set forth in references. Materials, reagents and the like to which reference are made in the following description and examples are obtainable from commercial sources, unless otherwise noted. General synthetic procedures have been described in treatise such as Fieser and Fieser's Reagents for Organic Synthesis; Wiley & Sons: New York, Volumes 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40 and will be familiar to those skilled in the art.

In one embodiment of the present invention there is provided a compound according to formula I wherein R¹, R², R³ and R⁴ are as described herein above.

In a second embodiment of the present invention there is provided a compound according to formula Ia wherein R^{1a} is optionally substituted p-phenylene, R^{1b} is NR^{a}R^{b}, R^{a} is hydrogen and R^{b} is hydroxy-C₁₋₆ alkanoyl, C₁₋₆ acyl, optionally substituted phenylcarbonyl or optionally substituted heteroarylcarbonyl

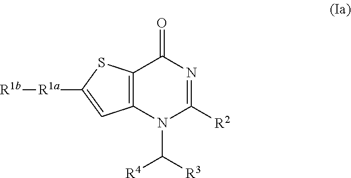

In a third embodiment of the present invention there is provided a compound according to formula Ia wherein R^{1a} is p-phenylene optionally further substituted by halogen, R^{1b} is NR^{a}R^{b}, R² and R⁴ are hydrogen R³ is phenyl optionally substituted by halogen or C₁₋₆ alkyl, R^{a} is hydrogen and R^{b} is hydroxy-C₁₋₆ alkanoyl, C₁₋₆ acyl, optionally substituted phenylcarbonyl or optionally substituted heteroarylcarbonyl.

In a fourth embodiment of the present invention there is provided a compound according to formula Ia wherein R^{1a} is p-phenylene optionally further substituted by halogen, R^{1b} is NR^{a}R^{b}, R² and R⁴ are hydrogen, R³ is phenyl optionally substituted by halogen or C₁₋₆ alkyl, R^{a} is hydrogen and R^{b} is optionally substituted phenylcarbonyl or optionally substituted heteroarylcarbonyl.

In a fifth embodiment of the present invention there is provided a compound according, to formula Ia wherein R^{1a} is optionally substituted p-phenylene, R^{1b} is optionally substituted heteroaryl and R² and R⁴ are hydrogen.

In a sixth embodiment of the present invention there is provided a compound according to formula Ia wherein R^{1a} is optionally substituted p-phenylene, R^{1b} is optionally substituted pyrazolo[1,5-a]pyrimidin-2-yl, R³ is phenyl optionally substituted by halogen or C₁₋₆ alkyl and R² and R⁴ are hydrogen.

In a seventh embodiment of the present invention there is provided a compound according to formula Ia wherein R^{1a} is optionally substituted p-phenylene, R^{1b} is 7-amino-5-methyl-pyrazolo[1,5-a]pyrimidin-2-yl, R³ is phenyl optionally substituted by halogen or C₁₋₆ alkyl and R² and R⁴ are hydrogen.

In another embodiment of the present invention there is provided a compound according to formula Ia wherein R^{1a} is optionally substituted p-phenylene, R^{1b} is optionally substituted pyrimidinyl, R³ is phenyl optionally substituted by halogen or C₁₋₆ alkyl and R² and R⁴ are hydrogen.

In an eighth embodiment of the present invention there is provided a compound according to formula Ia wherein R^{1a} is optionally substituted p-phenylene and R^{1b} is optionally substituted phenyl-C₁₋₃ alkoxy or optionally substituted heteroaryl-methoxy.

In a ninth embodiment of the present invention there is provided a compound according to formula Ia wherein R^{1a} is optionally substituted p-phenylene and R^{1b} is optionally substituted benzyloxy and R² and R⁴ are hydrogen.

In a another embodiment of the present invention there is provided a compound according to formula Ia wherein R^{1a} is optionally substituted p-pyridinylene and R^{1b} is optionally substituted benzyloxy and R² and R⁴ are hydrogen.

In a tenth embodiment there is provided a compound selected from I-1 to I-59 in TABLE I.

In a eleventh embodiment of the present invention there is provide a method of treating a HCV infection in a patient in need thereof comprising administering a therapeutically effective amount of a compound according to formula I wherein R¹, R², R³ and R⁴ are as defined hereinabove.

In a twelfth embodiment of the present invention there is provide a method of treating a HCV infection in a patient in need thereof comprising co-administering a therapeutically effective amount of a compound according to formula I wherein R¹, R², R³ and R⁴ are as defined herein above and at least one immune system modulator and/or at least one antiviral agent that inhibits replication of HCV.

In a thirteenth embodiment of the present invention there is provide a method of treating a disease caused by HCV in a patient in need thereof comprising co-administering a therapeutically effective amount of a compound according to formula I wherein R¹, R², R³ and R⁴ are as defined herein above and at least one immune system modulator selected from interferon, interleukin, tumor necrosis factor or colony stimulating factor.

In a fourteenth embodiment of the present invention there is provide a method of treating a HCV infection in a patient in need thereof comprising co-administering a therapeutically effective amount of a compound according to formula I wherein R¹, R², R³ and R⁴ are as defined herein above and an interferon or chemically derivatized interferon.

In a fifteenth embodiment of the present invention there is provide a method of treating a HCV infection in a patient in need thereof comprising co-administering a therapeutically effective amount of a compound according to formula I wherein R¹, R², R³ and R⁴ are as defined herein above and another antiviral compound selected from the group consisting of a HCV protease inhibitor, another HCV polymerase inhibitor, a HCV helicase inhibitor, a HCV primase inhibitor and a HCV fusion inhibitor.

In a sixteenth embodiment of the present invention there is provided a method for inhibiting viral replication in a cell by delivering a therapeutically effective amount of a compound of the formula I wherein R¹, R², R³ and R⁴ are as defined herein above admixed with at least one pharmaceutically acceptable carrier, diluent or excipient.

In a seventeenth embodiment of the present invention there is provided a composition comprising a compound according to formula I wherein R¹, R², R³ and R⁴ are as defined herein above admixed with at least one pharmaceutically acceptable carrier, diluent or excipient.

The term “alkyl” as used herein without further limitation alone or in combination with other groups, denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₆ alkyl” as used herein refers to an alkyl composed of 1 to 6 carbons. Examples of alkyl groups include, but are not limited to, lower alkyl groups include methyl, ethyl, propyl, iso-propyl, n-butyl, iso-butyl, tert-butyl, neopentyl, hexyl, and octyl. Any carbon hydrogen bond can be replaced by a carbon deuterium bond with departing from the scope of the invention.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to a substituent that can be either an aryl or a heteroaryl group.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “haloalkyl” as used herein denotes a unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, 1-fluoroethyl, 1-chloroethyl, 12-fluoroethyl, 2-chloroethyl, 2-bromoethyl, 2,2-dichloroethyl, 3-bromopropyl or 2,2,2-trifluoroethyl. The term “fluoroalkyl” as used herein refers to a haloalkyl moiety wherein fluorine is the halogen.

The term “aryl” as used herein refers to phenyl ring which can optionally be substituted with one to three substituents independently selected from hydroxy, cyano, C₁₋₆ alkyl, C₁₋₆ alkoxy, halogen, haloalkyl, nitro, alkoxycarbonyl, amino, alkylamino, dialkylamino, unless otherwise indicated.

The term “benzyl” as used herein refers to a C₆H₅CH₂ radical wherein the phenyl ring is can optionally be substituted with one to three substituents as described above for aryl, unless otherwise indicated.

The term “phenoxymethyl” as used herein refers to a PhOCH₂— radical wherein the phenyl ring is can optionally be substituted with one to three substituents as described above for aryl, unless otherwise indicated.

The term “halogen” or “halo” as used herein means fluorine, chlorine, bromine, or iodine.

The terms “hydroxyalkyl” and “alkoxyalkyl” as used herein denotes alkyl radical as herein defined wherein one to three hydrogen atoms on different carbon atoms is/are replaced by hydroxyl or alkoxy groups respectively. A C₁₋₃ alkoxy-C₁₋₆ alkyl moiety refers to a C₁₋₆ alkyl substituent in which 1 to 3 hydrogen atoms are replaced by a C₁₋₃ alkoxy and the point of attachment of the alkoxy is the oxygen atom.

The term “cyano” as used herein refers to a carbon linked to a nitrogen by a triple bond, i.e., —C≡N. The term “nitro” as used herein refers to a group —NO₂. The term “carboxy” as used herein refers to a group —CO₂H.

The term “acyl” (or “alkanoyl”) as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl or “alkanoyl” refers to a group —C(═O)R contain 1 to 6 carbon atoms. The Cᵢ acyl group is the formyl group wherein R═H and a C6 acyl group refers to hexanoyl when the alkyl chain is unbranched. The term “arylcarbonyl” or “aroyl” as used herein means a group of formula C(═O)R wherein R is an aryl group. The term “heteroarylcarbonyl” or “heteroaroyl” as used herein means a group of formula C(═O)R wherein R is an heteroaryl group. The term “benzoyl” or “phenylcarbonyl” as used herein is an “arylcarbonyl” or “aroyl” group wherein R is phenyl.

The terms “amino”, “alkylamino” and “dialkylamino” as used herein refer to —NH₂, —NHR and —NR₂ respectively and R is alkyl as defined above. The two alkyl groups attached to a nitrogen in a dialkyl moiety can be the same or different. The terms “aminoalkyl”, “alkylaminoalkyl” and “dialkylaminoalkyl” as used herein refer to NH₂(CH₂)ₙ—, RHN(CH₂)ₙ—, and R₂N(CH₂)ₙ— respectively wherein n is 1 to 6 and R is alkyl as defined above. “C₁₋₁₀ alkylamino” as used herein refers to an alkylamino moiety wherein alkyl is C₁₋₁₀. The term “phenylamino” as used herein refers to —NHPh wherein Ph represents an optionally substituted phenyl group.

The term “cyclic amine” as used herein refers to a saturated carbon ring, containing from 3 to 6 carbon atoms as defined above, and wherein at least one of the carbon atoms is replaced by a heteroatom selected from the group consisting of N, O and S, for example, piperidine, piperazine, morpholine, thiomorpholine, di-oxo-thiomorpholine, pyrrolidine, pyrazoline, imidazolidine, azetidine wherein the cyclic carbon atoms are optionally substituted by one or more substituents, selected from the group consisting of halogen, hydroxy, phenyl, lower alkyl, lower alkoxy or 2-hydrogen atoms on a carbon are both replace by oxo (═O). When the cyclic amine is a piperazine, one nitrogen atom can be optionally substituted by C₁₋₆ alkyl, C₁₋₆ acyl, C₁₋₆ alkylsulfonyl.

The terms “alkylsulfonyl” and “arylsulfonyl” as used herein denotes a group of formula —S(═O)₂R wherein R is alkyl or aryl respectively and alkyl and aryl are as defined herein. The term C₁₋₃ alkylsulfonylamido as used herein refers to a group RSO₂NH— wherein R is a C₁₋₃ alkyl group as defined herein. The terms C₁₋₆ haloalkylsulfonyl, C₃₋₇ cycloalkylsulfonyl, C₃₋₇ cycloalkyl-C₁₋₃ alkyl-sulfonyl or C₁₋₆ alkoxy-C₁₋₆ alkylsulfonyl refer to a compound, S(═O)₂R wherein R is C₁₋₆ haloalkyl, C₃₋₇ cycloalkyl, C₃₋₇ cycloalkyl-C₁₋₃ alkyl and C₁₋₆ alkoxy-C₁₋₆ alkyl, respectively.

The term “pyridinylmethylsulfanyl” as used herein refers to a moiety (pyrindinyl)CH₂S—. The term “phenyl-C₁₋₃ alkylsulfanyl” refers to a moiety PhCH₂S—.

The term “heteroaryl C₁₋₃ alkoxy” as used herein alkoxy radicel as defined herein wherein a hydrogen on the alkoxy group is replaced by a heteroaryl group with the understanding that the attachment point of the “heteroaryl C₁₋₃ alkoxy” moiety will at the oxygen atom of the alkoxy group.

The term “4-(pyrazolo[1,5-a]pyrimidin-2-yl)-” refers to the following moiety.

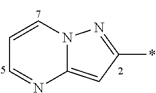

The term “phenylene” as used herein refers to a benzene ring with two open valences. A phenylene moiety has three possible regioisomers, ortho-, -meta- or para-phenylene. The phase “optionally substituted p-phenylene” as used herein refers to a p-phenylene moiety wherein one of the remaining hydrogens attached to carbon can optionally be replace by a substituent. The term “pyridinylene” as used herein refers to a pyridine ring with two open valences. The term p-pyridinylene moiety has two regioisomers (i) and (ii) wherein A and B are different.

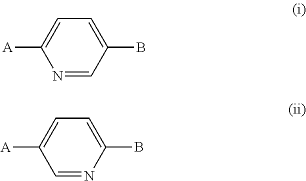

Compounds of the present invention and their isomeric forms and pharmaceutically acceptable salts thereof are also useful in treating viral infections, in particular, hepatitis C infection, and diseases in living hosts when used in combination with each other and with other biologically active agents, including but not limited to the group consisting of interferon, a pegylated interferon, ribavirin, protease inhibitors, polymerase inhibitors, small interfering RNA compounds, antisense compounds, nucleotide analogs, nucleoside analogs, immunoglobulins, immunomodulators, hepatoprotectants, anti-inflammatory agents, antibiotics, antivirals and anti-infective compounds. Such combination therapy may also comprise providing a compound of the invention either concurrently or sequentially with other medicinal agents or potentiators, such as ribavirin and related compounds, amantadine and related compounds, various interferons such as, for example, interferon-alpha, interferon-beta, interferon gamma and the like, as well as alternate forms of interferons such as pegylated interferons. Additionally combinations of ribavirin and interferon, may be administered as an additional combination therapy with at least one of the compounds of the present invention.

In one embodiment, the compounds of the present invention according to formula I are used in combination with other active therapeutic ingredients or agents to treat patients with an HCV viral infection. According to the present invention, the active therapeutic ingredient used in combination with the compound of the present invention can be any agent having a therapeutic effect when used in combination with the compound of the present invention. For example, the active agent used in combination with the compound of the present invention can be interferons, ribavirin analogs, HCV NS3 protease inhibitors, nucleoside inhibitors of HCV polymerase, non-nucleoside inhibitors of HCV polymerase, and other drugs for treating HCV, or mixtures thereof.

Examples of the nucleoside NS5b polymerase inhibitors include, but are not limited to NM-283, valopicitabine, R1626, PSI-6130 (R1656), IDX184 and IDX102 (Idenix) BILB 1941.

Examples of the non-nucleoside NS5b polymerase inhibitors include, but are not limited to HCV-796 (ViroPharma and Wyeth), MK-0608, MK-3281 (Merck), NM-107, R7128 (R4048), VCH-759, GSK625433 and GSK625433 (Glaxo), PF-868554 (Pfizer), GS-9190 (Gilead), A-837093 and A848837 (Abbot Laboratories), ANA598 (Anadys Pharmaceuticals); GL100597 (GNLB/NVS), VBY 708 (ViroBay), benzimidazole derivatives (H. Hashimoto et al. WO 01/47833, H. Hashimoto et al. WO 03/000254, P. L. Beaulieu et al. WO 03/020240 A2; P. L. Beaulieu et al. U.S. Pat. No. 6,448,281 B1; P. L. Beaulieu et al. WO 03/007945 A1), benzo-1,2,4-thiadiazine derivatives (D. Dhanak et al. WO 01/85172 A1, filed May 10, 2001; D. Chai et al., WO2002098424, filed Jun. 7, 2002, D. Dhanak et al. WO 03/037262 A2, filed Oct. 28, 2002; K. J. Duffy et al. WO03/099801 A1, filed May 23, 2003, M. G. Darcy et al. WO2003059356, filed Oct. 28, 2002; D. Chai et al. WO 2004052312, filed Jun. 24, 2004, D. Chai et al. WO2004052313, filed Dec. 13, 2003; D. M. Fitch et al., WO2004058150, filed Dec. 11, 2003; D. K. Hutchinson et al. WO2005019191, filed Aug. 19, 2004; J. K. Pratt et al. WO 2004/041818 A1, filed Oct. 31, 2003), 1,1-dioxo-4H-benzo[1,4]thiazin-3-yl derivatives (J. F. Blake et al. in U.S. Patent Publication US20060252785 and 1,1-dioxo-benzo[d]isothazol-3-yl compounds (J. F. Blake et al. in U.S. Patent Publication 2006040927).

Examples of the HCV NS3 protease inhibitors include, but are not limited to SCH-503034 (Schering, SCH-7), VX-950 (telaprevir, Vertex), BILN-2065 (Boehringer-Ingelheim, BMS-605339 (Bristol Myers Squibb), and ITMN-191 (Intermune).

Examples of the interferons include, but are not limited to pegylated rIFN-alpha 2b, pegylated rIFN-alpha 2a, rIFN-alpha 2b, rIFN-alpha 2a, consensus IFN alpha (infergen), feron, reaferon, intermax alpha, r-IFN-beta, infergen and actimmune, IFN-omega with DUROS, albuferon, locteron, Albuferon, Rebif, oral interferon alpha, IFNalpha-2b XL, AVI-005, PEG-Infergen, and pegylated IFN-beta.

Ribavirin analogs and the ribavirin prodrug viramidine (taribavirin) have been administered with interferons to control HCV.

Commonly used abbreviations include: acetyl (Ac), aqueous (aq.), atmospheres (Atm), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), methanol (MeOH), melting point (mp), MeSO₂— (mesyl or Ms), methyl (Me), acetonitrile (MeCN), m-chloroperbenzoic acid (MCPBA), mass spectrum (ms), methyl tent-butyl ether (MTBE), N-methylmorpholine (NMM), N-methylpyrrolidone (NMP), phenyl (Ph), propyl (Pr), iso-propyl (i-Pr), pounds per square inch (psi), pyridine (pyr), room temperature (rt or RT), satd. (saturated), tert-butyldimethylsilyl or t-BuMe₂Si (TBDMS), triethylamine (TEA or Et₃N), triflate or CF₃SO₂— (TO, trifluoroacetic acid (TFA), O-benzotriazol-1-yl-N,N,N′,N′-tetramethyluronium tetrafluoroborate (TBTU), thin layer chromatography (TLC), tetrahydrofuran (THF), tetramethylethylenediamine (TMEDA), trimethylsilyl or Me₃Si (TMS), p-toluenesulfonic acid monohydrate (TsOH or pTsOH), 4-Me—C₆H₄SO₂— or tosyl (Ts), N-urethane-N-carboxyanhydride (UNCA). Conventional nomenclature including the prefixes normal (n-), iso (i-), secondary (sec-), tertiary (tent-) and neo- have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The following number scheme is employed to describe compounds of the present invention.

TABLE I

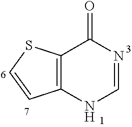

| | | | | IC₅₀ |
|---|---|---|---|---|
| Cpd. | | | | HCV |
| No. | Structure | ms | mp | pol¹ |
| I-1 | 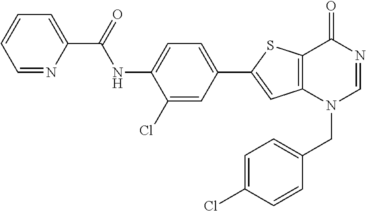 | 507/509 | | 0.002 |
| I-2 | 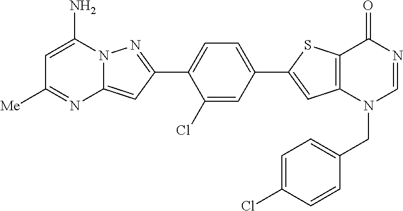 | 533/535 | | 0.004 |
| I-3 | 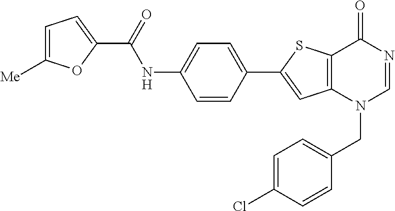 | 476/478 | | 0.009 |
| I-4 | 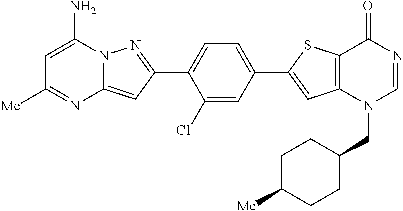 | 519/521 | | 0.010 |
| I-5 | 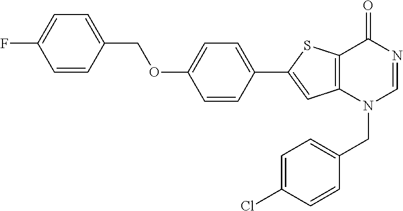 | 477/479 | | 0.013 |
| I-6 | 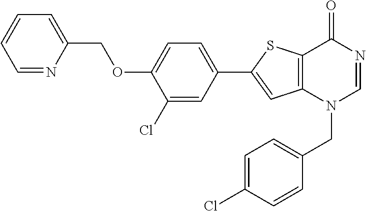 | 494/496 | 272-274 | 0.018 |
| I-7 | 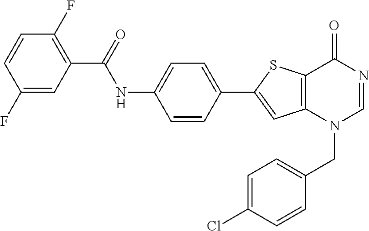 | 508/510 | | 0.021 |
| I-8 | 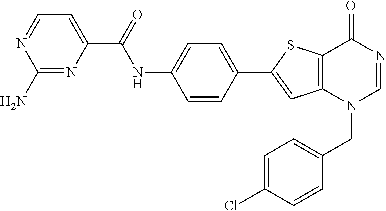 | 489/491 | | 0.026 |
| I-9 | 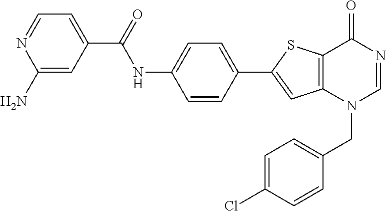 | 488/490 | | 0.029 |
| I-10 | 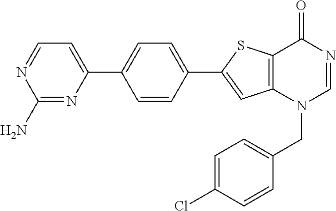 | 446/448 | | 0.029 |
| I-11 | 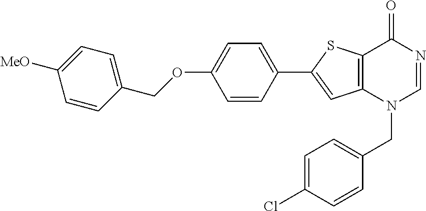 | 489/491 | | 0.032 |
| I-12 | 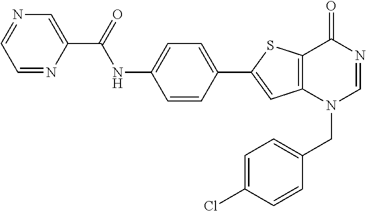 | 474/476 | | 0.032 |
| I-13 | 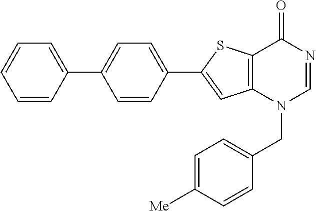 | 409 | | 0.045 |
| I-14 | 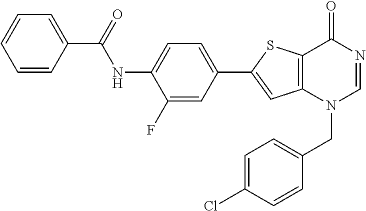 | 490/492 | | 0.069 |
| I-15 | 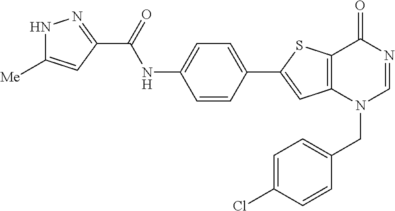 | 476/478 | | 0.070 |
| I-16 | 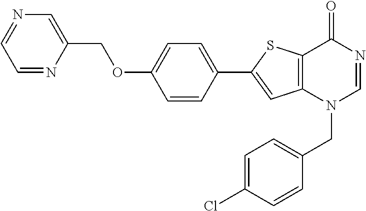 | 461/463 | 236.9-239.6 | 0.071 |
| I-17 | 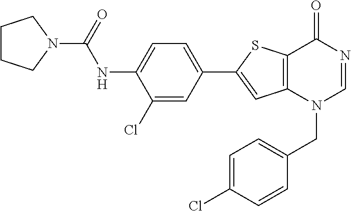 | 490/492 | | 0.076 |
| I-18 | 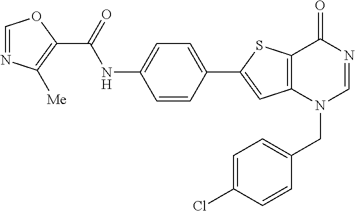 | 477/479 | | 0.077 |
| I-19 | 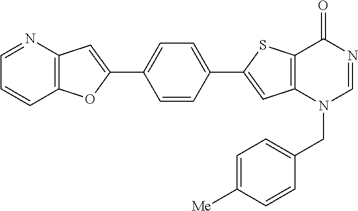 | 450 | | 0.082 |
| I-20 | 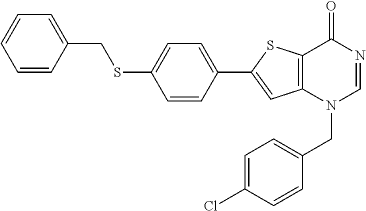 | 475/477 | | 0.104 |
| I-21 | 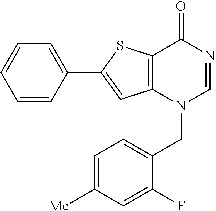 | 351 | 225.1-225.8 | 0.126 |
| I-22 | 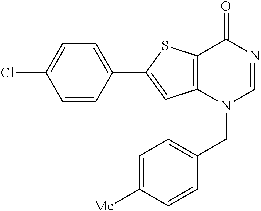 | 367/369 | 244.1-247.5 | 0.131 |
| I-23 | 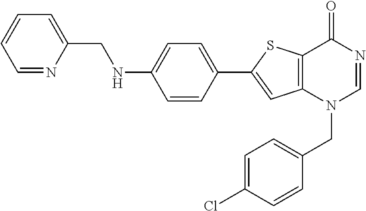 | 459/461 | | 0.139 |
| I-24 | 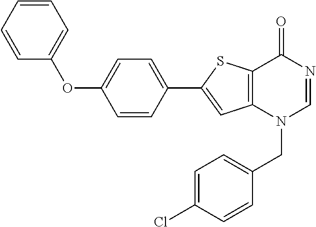 | 445/447 | | 0.150 |
| I-25 | 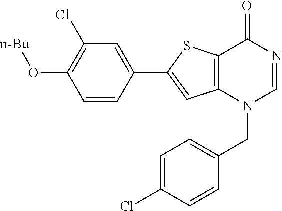 | 445/447 | | 0.155 |
| I-26 | 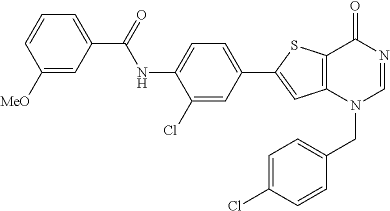 | 536/539 | | 0.162 |
| I-27 | 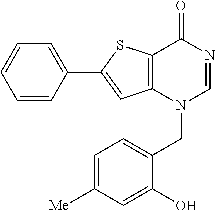 | 346 | 266.7-267.7 | 0.202 |
| I-28 | 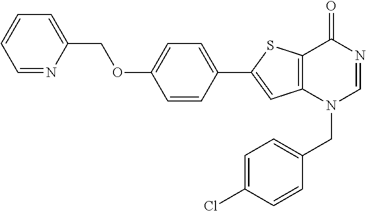 | 460/462 | 233-235 | 0.051 |
| I-29 | 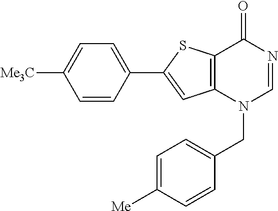 | 389 | 260.5-261.5 | 0.223 |
| I-30 | 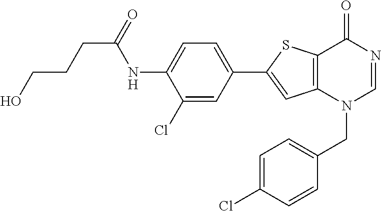 | 488/490 | | 0.279 |
| I-31 | 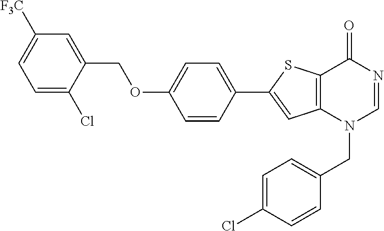 | 561/563 | | 0.296 |
| I-32 | 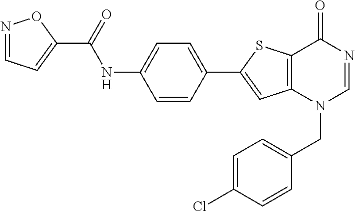 | 463/465 | | 0.308 |
| I-33 | 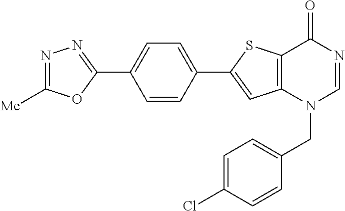 | 465/467 | | 0.390 |
| I-34 | 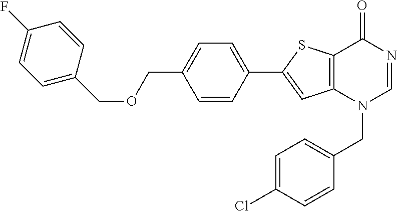 | 491/493 | | 0.514 |
| I-35 | 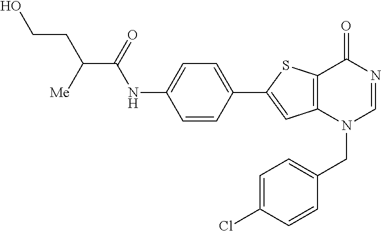 | 502/504 | | 0.517 |
| I-36 | 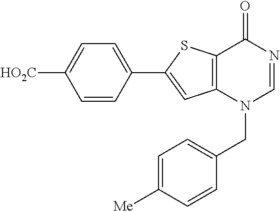 | 377 | | 0.535 |
| I-37 | 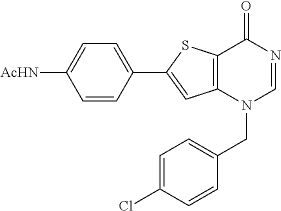 | 410/412 | | 0.586 |
| I-38 | 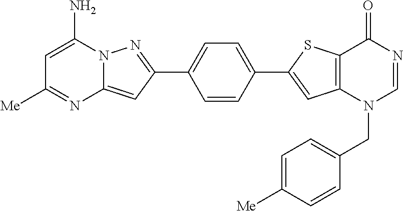 | 479 | | 0.21² |
| I-39 | 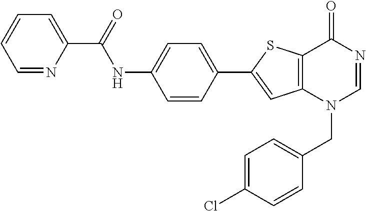 | 476/475 | | 0.005 |
| I-40 | 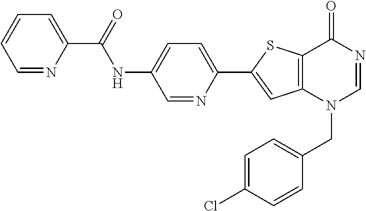 | 474/476 | | 0.131 |
| I-41 | 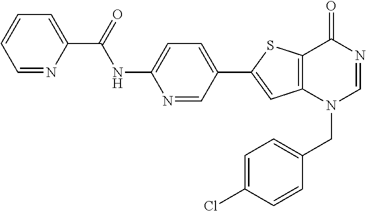 | 474/476 | | 0.062 |
| I-42 | 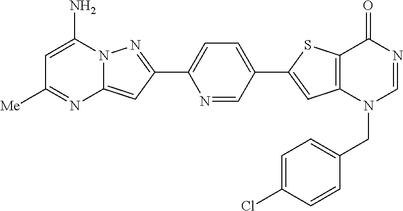 | 500/502 | | 0.019 |
| I-43 | 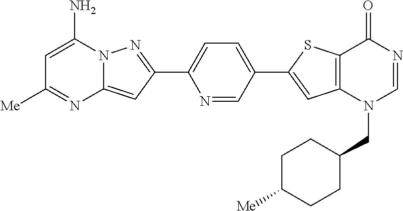 | 486/488 | | 0.428 |
| I-44 | 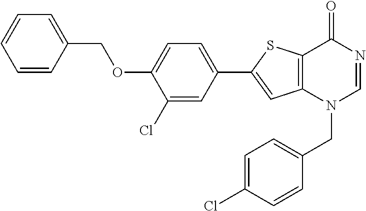 | 493/495 | 222.0-224.0 | 0.03 |
| I-45 | 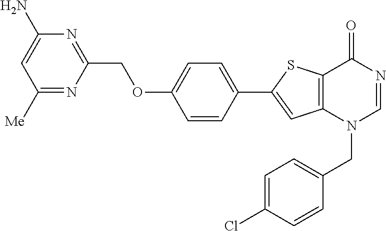 | 490/492 | 272.0-274.0 | 0.372 |
| I-46 | 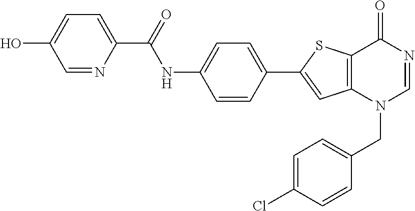 | 489/491 | | 0.017 |
| I-47 | 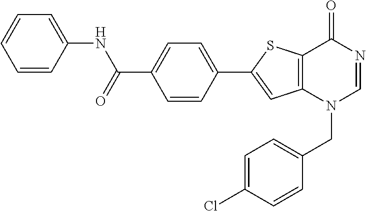 | 472/474 | | ca. 0.040 |
| I-48 | 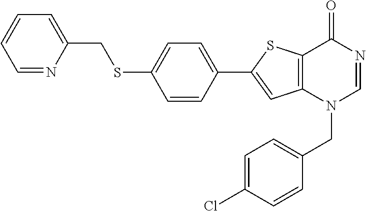 | 476/478 | | 0.163 |
| I-49 | 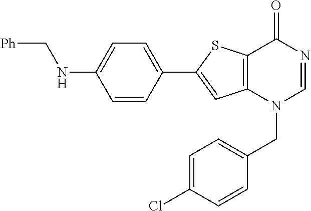 | 458/460 | | 0.377 |
| I-50 | 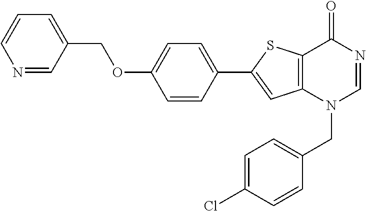 | 460/462 | 255-257 | 0.103 |
| I-51 | 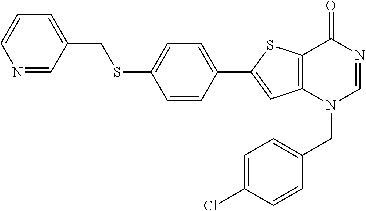 | 476/478 | | 0.313 |
| I-52 | 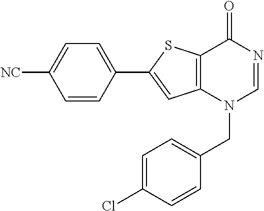 | 378 | >300 | 1.6 |
| I-53 | 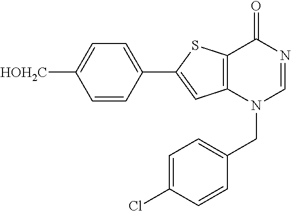 | 363 | | 0.56 |
| I-54 | 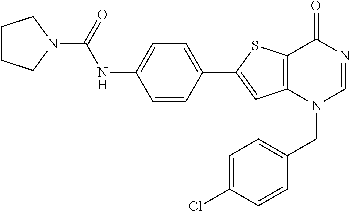 | 499/501 | | 0.076 |
| I-55 | 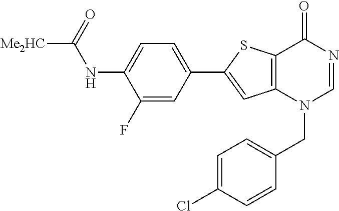 | 456/458 | | 1.79 |
| I-56 | 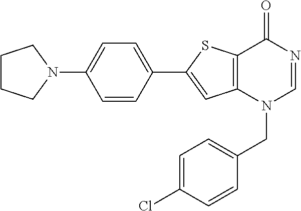 | 422/424 | | 1.61 |
| I-57 | 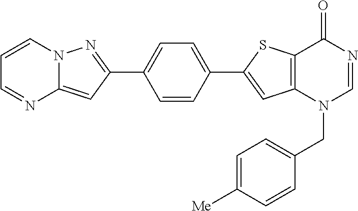 | 450 | | 0.108² |
| I-58 | 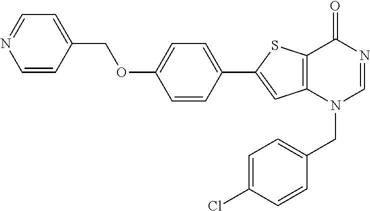 | 460/462 | 257-259 | 0.049 |
| I-59 | 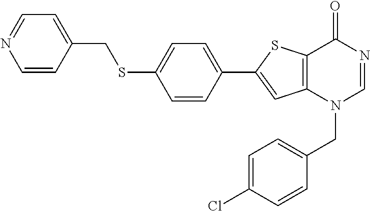 | 476/478 | | 0.167 |

¹see example 20 primer the oligo(rU)16/poly A terminated template
²as in example 20 but using the cIRES terminated template

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents forOrganic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Some compounds in following schemes are depicted with generalized substituents; however, one skilled in the art will immediately appreciate that the nature of the R groups can varied to afford the various compounds contemplated in this invention. Moreover, the reaction conditions are exemplary and alternative conditions are well known. The reaction sequences in the following examples are not meant to limit the scope of the invention as set forth in the claims

The 1-N-substituted-6-(hetero)aryl-1H-thieno[3,2-d]pyrimidin-4-one scaffold common to compounds of the present invention is prepared from methyl 3-amino-5-bromothiophene-1-carboxylate by alkylation of the amine by reductive amination to afford A-1b which is cyclized with formamidine to afford A-2. Introduction of the C-7 moiety is carried out using palladium-catalyzed coupling chemistry to afford A-3.

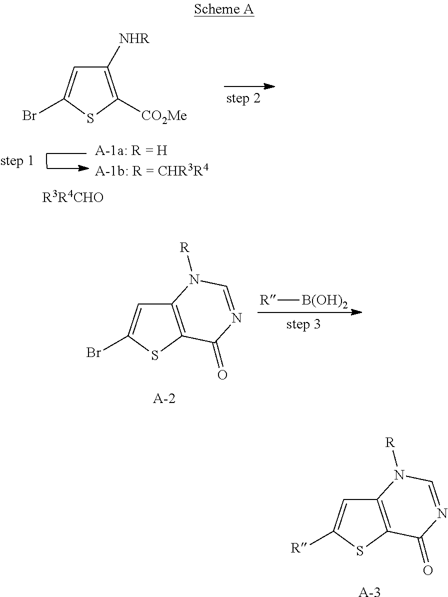

When R³ is optionally substituted phenyl and R⁴ is hydrogen, step 1 is carried out with an optionally substituted benzaldehyde. The corresponding compound wherein R⁴ is alkyl can be prepared from the corresponding alkyl phenyl ketone. When R³ is optionally substituted cycloalkyl the reductive amination is carried out with a cycloalkylcarboxaldehyde.

Reductive amination is typically carried out by combining an amine and carbonyl compound in the presence of a complex metal hydride such as NaBH₄, LiBH₄, NaBH₃CN, Zn(BH₄)₂, sodium triacetoxyborohydride or borane/pyridine conveniently at a pH of 1-7 optionally in the presence of a dehydrating agent such as molecular sieve or Ti(IV)(O-i-Pr)₄ to facilitate formation of the intermediate imine at ambient temperature. Alternatively the imine can be formed under a hydrogen atmosphere in the presence of a hydrogenation catalyst, e.g. in the presence of Pd/C, at a hydrogen pressure of 1 to 5 bar, preferably at temperatures between 20° C. and the boiling temperature of the solvent used. It may also be advantageous during the reaction if reactive groups are protected during the reaction by conventional protecting groups which are cleaved again by conventional methods after the reaction. Reductive amination procedures have been reviewed: R. M. Hutchings and M. K. Hutchings Reduction of C═N to CHNH by Metal Hydrides in Comprehensive Organic Synthesis col. 8, I. Fleming (Ed) Pergamon, Oxford 1991 pp. 47-54.

The 7-(hetero)aryl substituent is introduced utilizing a palladium-catalyzed Suzuki coupling of A-2 and an optionally substituted (hetero)arylboronic acid. One skilled in the art will appreciate that substituted (hetero)aryl boronic acids are readily available and the Suzuki coupling of A-3 in step 3 may directly result in the desired product or the initially introduced (hetero)aryl moiety may be further modified. Thus, as described in example 1, if the boronic acid is [4-(tert-butoxycarbonylamino)-phenyl]boronic acid, removal of the Boc group affords an amine that can be further functionalized by e.g, acylation or alkylation of the unmasked amine. Representative examples of subsequent transformation the Suzuki coupling product are disclosed in the examples that follow.

The Suzuki reaction is a palladium-catalyzed coupling of a boronic acid (R—B(OH)₂) wherein R is aryl or vinyl) with an aryl or vinyl halide or triflate (R′Y wherein R′=aryl or vinyl; Y=halide or OSO₂CF₃) to afford a compound R-R′. Typical catalysts include Pd(PPh₃)₃, Pd(OAc)₂ and PdCl₂(dppf). With PdCl₂(dppf), primary alkyl borane compounds can be coupled to aryl or vinyl halide or triflate without β-elimination. Highly active catalysts have been identified (see, e.g. J. P. Wolfe et al., J. Am. Chem. Soc. 1999 121(41):9550-9561 and A. F. Littke et al., J. Am. Chem. Soc. 2000 122(17):4020-4028). The reaction can be carried out in a variety of organic solvents including toluene, THF, dioxane, 1,2-dichloroethane, DMF, DMSO and acetonitrile, aqueous solvents and under biphasic conditions. Reactions are typically run from about room temperature to about 150° C. Additives (e.g. CsF, KF, TlOH, NaOEt and KOH) frequently accelerate the coupling. There are a large number of parameters in the Suzuki reaction including the palladium source, ligand, additives and temperature and optimum conditions sometimes require optimization of the parameters for a given pair of reactants. A. F. Littke et al., supra, disclose conditions for Suzuki cross-coupling with arylboronic acids in high yield at RT utilizing Pd₂(dba)₃/P(tert-Bu)₃ and conditions for cross-coupling of aryl- and vinyl triflates utilizing Pd(OAc)₂/P(C₆H₁₁)₃ at RT. J. P. Wolf et al., supra, disclose efficient condition for Suzuki cross-coupling utilizing Pd(OAc)₂%-(di-tert-butylphosphino)biphenyl or o-(dicyclohexylyphosphino)biphenyl. One skilled in the art can determine optimal conditions without undue experimentation.

Acylation of the amine is conveniently carried out with an acyl halide or acid anhydride in a solvent such as DCM, CHCl₃, carbon tetrachloride, ether, THF, dioxane, benzene, toluene, MeCN, DMF, aqueous NaOH solution or sulfolane optionally in the presence of an inorganic or organic base at temperatures between −20 and 200° C., but preferably at temperatures between −10 and 160° C. Typical organic bases include tertiary amines include but are not limited to TEA, pyridine. Typical inorganic bases include but are not limited to K₂CO₃ and NaHCO₃.

The acylation may however also be carried out with the free acid optionally in the presence of an acid-activating agent or a dehydrating agent, e.g. in the presence of isobutyl chloroformate, thionyl chloride, trimethylchlorosilane, hydrogen chloride, sulphuric acid, methanesulphonic acid, p-TsOH, PCl₃, P₂O₅, DCC, DCC/N-hydroxysuccinimide or HOBt, CDI, O-(benzotriazol-1-yl)-N,N,N′,N′-tetramethyl-uronium tetrafluoroborate/NMM, O-(benzotriazol-1-yl)-N,N,N′,N′-tetramethyl-uronium tetrafluoroborate/DIPEA, N,N′-thionyldiimidazole or PPh₃/CCl₄, at temperatures between −20 and 200° C., but preferably at temperatures between −10 and 160° C.

Substituted amines and sulfanes can be prepared analogously by alkylation of an amine or a thiophenol.

Compounds encompassed by the present invention wherein R″ is phenyl ether can be prepared by coupling of the appropriately substituted boronic acid (e.g., example 8). Alkylation of a phenolic boronic acid such as 4-(4,4,5,5-tetramethyl-[1,3,2]dioxaborolan-2-yl)-phenol affords a variety of structurally diverse boronic acids that can be employed to produce the compounds described herein (e.g., example 10).

Alkylation of phenols is typically carried out in solvents like DMF, THF, NMP, MeCN, acetone, DCM and DCE, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates. Alternate procedures such as the Mitsunobu coupling are well know in the art and can be used where advantageous. Analogous alkylation of thiol or amine can be carried out under similar conditions.

Compounds encompassed by the present invention wherein R″ is a phenyl ring substituted with a heteroaryl moiety are prepared by palladium-catalyzed coupling of A-2 and a boronic acid such as 2-[4-(4,4,5,5-tetramethyl-[1,3,2]dioxaborolan-2-yl)-phenyl]-pyrazolo[1,5-a]pyrimidine or derivatives thereof which are known in the art.

### Antiviral Activity

The activity of the inventive compounds as inhibitors of HCV activity may be measured by any of the suitable methods known to those skilled in the art, including in vivo and in vitro assays. For example, the HCV NS5B inhibitory activity of the compounds of formula I can determined using standard assay procedures described in Behrens et al., EMBO J. 1996 15:12-22, Lohmann et al., Virology 1998 249:108-118 and Ranjith-Kumar et al., J. Virology 2001 75:8615-8623. Unless otherwise noted, the compounds of this invention have demonstrated in vitro HCV NS5B inhibitory activity in such standard assays. The HCV polymerase assay conditions used for compounds of the present invention are described in Example 20. Cell-based replicon systems for HCV have been developed, in which the nonstructural proteins stably replicate subgenomic viral RNA in Huh7 cells (V. Lohmann et al., Science 1999 285:110 and K. J. Blight et al., Science 2000 290:1972. The cell-based replicon assay conditions used for compounds of the present invention are described in Example 21. In the absence of a purified, functional HCV replicase consisting of viral non-structural and host proteins, our understanding of Flaviviridae RNA synthesis comes from studies using active recombinant RNA-dependent RNA-polymerases and validation of these studies in the HCV replicon system Inhibition of recombinant purified HCV polymerase with compounds in vitro biochemical assays may be validated using the replicon system whereby the polymerase exists within a replicase complex, associated with other viral and cellular polypeptides in appropriate stoichiometry. Demonstration of cell-based inhibition of HCV replication may be more predictive of in vivo function than demonstration of HCV NS5B inhibitory activity in vitro biochemical assays.

### Dosage and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent such as ribavirin, a nucleoside HCV polymerase inhibitor, another HCV non-nucleoside polymerase inhibitor or HCV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

A therapeutically effective amount of a compound of the present invention, and optionally one or more additional antiviral agents, is an amount effective to reduce the viral load or achieve a sustained viral response to therapy. Useful indicators for a sustained response, in addition to the viral load include, but are not limited to liver fibrosis, elevation in serum transaminase levels and necroinflammatory activity in the liver. One common example, which is intended to be exemplary and not limiting, of a marker is serum alanine transminase (ALT) which is measured by standard clinical assays. In some embodiments of the invention an effective treatment regimen is one which reduces ALT levels to less than about 45 IU/mL serum.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

In the experimental procedures which follow, the term “magic” as used herein relating to SiO₂ chromatography eluents refers to a 60/10/1 solution of DCM/MeOH/NH₄OH.

### Referential Example 1

### 6-Bromo-1-(4-methyl-benzyl)-1H-thieno[3,2-d]pyrimidin-4-one (30)

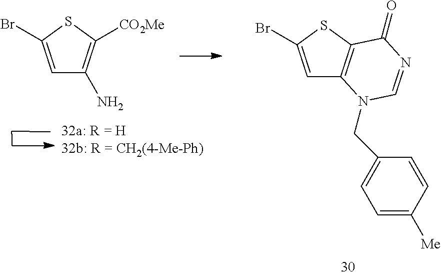

step 1—To a solution of 32a (1.58 g, 6.75 mmol, CASRN 07818-55-3) and 4-methyl-benzaldehyde (1 mL, 8.5 mmol) in DCM (20 mL) and HOAc (4 mL) cooled to 0° C. was added NaBH(OAc)₃ (1.6 g, 7.5 mmol). The reaction mixture was stirred and allowed to warm to RT. (In some cases additional aldehyde and NaBH(OAc)₃ were added to push the reaction to completion.) The reaction was cooled to 0° C., quenched with 2N NaOH, extracted with DCM, dried (Na₂SO₄), filtered and concentrated. The crude product was purified by SiO₂ chromatography eluting with an EtOAc/hexane gradient (2% to 5% EtOAc) to afford 1.65 g of 32b.

step 2—To a slurry of 32b (700 mg, 2.06 mmol) in formamide (12 mL) was added dropwise KO-tent-Bu (12 mL, 12 mmol, 1.0 M in THF). The reaction mixture was heated to reflux overnight, allowed to cool to RT, quenched by the addition of H₂O, extracted with DCM. The combined extracts were dried (Na₂SO₄), filtered and concentrated. The crude product was purified by SiO₂ chromatography eluting with a gradient consisting of a solution of DCM/MeOH/NH₄OH (60/10/1) (referred to herein as “Magic”) and DCM (98% to 85% DCM) to afford 0.5 g of 30.

In some cases, the cyclization did not go to completion. In these cases, mixtures of starting material and product were isolated, and subjected again to the cyclization conditions, to yield the desired product.

### Referential Example 2

### 6-Bromo-1-(4-chloro-benzyl)-1H-thieno[3,2-d]pyrimidin-4-one

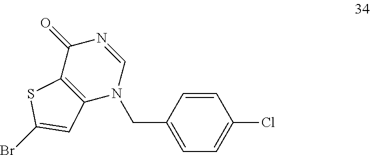

6-Bromo-1-(4-chloro-benzyl)-1H-thieno[3,2-d]pyrimidin-4-one (34) was synthesized in accord with the procedure described in Example 1, except 4-chloro-benzaldehyde was employed in the reductive amination step.

### Referential Example 3

### 6-Bromo-1-(trans-4-methyl-cyclohexylmethyl)-1H-thieno[3,2-d]pyrimidin-4-one (36)

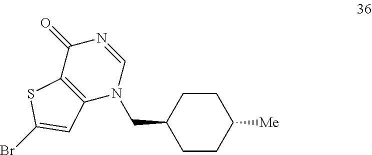

6-Bromo-1-(trans-4-methyl-cyclohexylmethyl)-1H-thieno[3,2-d]pyrimidin-4-one (36) was synthesized according to the procedure described in Example 1, except trans-4-methyl-cyclohexanecarbaldehyde was employed in the reductive amination step.

trans-4-Methyl-cyclohexanecarbaldehyde was synthesized starting from trans-4-methyl-cyclohexanecarboxylic acid. Methyl ester formation with TMSCHN₂, followed by Dibal reduction at −78° C., yielded trans-4-methyl-cyclohexanecarbaldehyde.

### Referential Example 4

### 6-Bromo-1-(2-fluoro-4-methyl-benzyl)-1H-thieno[3,2-d]pyrimidin-4-one (35)

6-Bromo-1-(2-fluoro-4-methyl-benzyl)-1H-thieno[3,2-d]pyrimidin-4-one (35) was synthesized according to the procedure described in Example 1, except 2-fluoro-4-methyl-benzaldehyde was employed in the reductive amination step.

### Example 1

### Pyridine-2-carboxylic acid {4-[1-(4-chloro-benzyl)-4-oxo-1,4-dihydro-thieno[3,2-d]pyrimidin-6-yl]-phenyl}-amide (I-39)

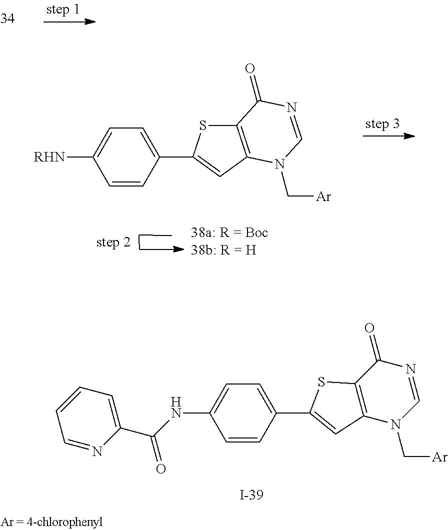

step 1—To a slurry of 34 (300 mg, 0.85 mmol), [4-(tert-butoxycarbonylamino)-phenyl]boronic acid (260 mg, 1.1 mmol) and Pd(PPh₃)₄ (125 mg) in DMF (5 mL) was added aq. sat'd. Na₂CO₃ (3 mL). The reaction mixture was stirred at 100° C. until all starting material was consumed (ca. 30 min). The reaction mixture was cooled to RT, quenched with H₂O, extracted with EtOAc, dried (Na₂SO₄) and concentrated. The crude product was purified by SiO₂ chromatography eluting with a Magic/DCM gradient (2% to 30% Magic) to afford 250 mg of 38a.

In some cases, after quenching the reaction mixture, the product precipitated after the reaction was quenched with H₂O. In these cases, the precipitate was collected, and the supernatant was extracted with EtOAc, dried (Na₂SO₄) and subjected to SiO₂ chromatography.

step 2—A solution of 38a (50 mg) and HCl-dioxane (1 mL, 4.0 M solution dioxane) was stirred at RT until all starting material was consumed. The solvent was evaporated to afford the hydrochloride salt of 38b which was used without further purification.

step 3—To a solution of 38b from step 2, pyridine-2-carboxylic acid (20 mg, 0.16 mmol) in DMF (1 mL) and TEA (0.05 mL) was added EEDQ (40 mg, 0.16 mmol). The reaction mixture was heated to 60° C. the reaction was complete, cooled to RT, and then quenched with H₂O. The resulting solid was collected to afford 10 mg of I-39: MS calcd for C₂₅H₁₇ClN₄O₂S [M+H]⁺ 473. Found, 473; ¹H NMR (DMSO-d₆, 300 MHz): δ 10.87 (broad s, 1H), 8.78-8.73 (m, 1H), 8.67 (s, 1H), 8.21-8.15 (m, 1H), 8.13-8.03 (m, 3H), 7.86-7.79 (m, 3H), 7.74-7.68 (m, 1H), 7.46 (s, 4H), 5.49 (s, 2H)

5-Hydroxy-pyridine-2-carboxylic acid, {4-[1-(4-chloro-benzyl)-4-oxo-1,4-dihydro-thieno[3,2-d]pyrimidin-6-yl]-phenyl}-amide (I-46) was prepared analogously except in step 3 pyridine-2-carboxyl acid was replaced with 5-hydroxy-pyridine-2-carboxylic acid (CASRN 15069-92-8) and EEDQ was replaced with HATU.

4-[1-(4-Chloro-benzyl)-4-oxo-1,4-dihydro-thieno[3,2-d]pyrimidin-6-yl]-N-phenyl-benzamide (I-47) was prepared analogously except in step 1, [4-(tert-butoxycarbonylamino)-phenyl]boronic acid was replaced with B-[4-[(phenylamino)carbonyl]phenyl]-boronic acid (CASRN 330793-45-8) and steps 2 and 3 were omitted.

6-Biphenyl-4-yl-1-(4-chloro-benzyl)-1H-thieno[3,2-d]pyrimidin-4-one (I-13) was prepared analogously except in step 1, [4-(tert-butoxycarbonylamino)-phenyl]boronic acid was replaced with B-(4-phenyl-phenyl)boronic acid (CASRN 5122-94-1) and steps 2 and 3 were omitted.

1-(4-Chloro-2-fluoro-benzyl)-6-phenyl-1H-thieno[3,2-d]pyrimidin-4-one (I-21) was prepared analogously except in step 1, [4-(tert-butoxycarbonylamino)-phenyl]boronic acid was replaced with phenylboronic acid (CASRN 98-80-6), 34 was replaced with 35 and steps 2 and 3 were omitted.

1-(4-Chloro-benzyl)-6-(4-chloro-phenyl)-1H-thieno[3,2-d]pyrimidin-4-one (I-22) was prepared analogously except in step 1, [4-(tert-butoxycarbonylamino)-phenyl]boronic acid was replaced with 4-chloro-phenylboronic acid (CASRN 1679-18-1), 34 was replaced with 30 and steps 2 and 3 were omitted.

1-(4-Chloro-benzyl)-6-(4-phenoxy-phenyl)-1H-thieno[3,2-d]pyrimidin-4-one (I-24) was prepared analogously except in step 1, [4-(tent-butoxycarbonylamino)-phenyl]boronic acid was replaced with 4-phenoxy-phenylboronic acid (CASRN 51067-38-0) and steps 2 and 3 were omitted.

6-(4-Butoxy-3-chloro-phenyl)-1-(4-chloro-benzyl)-1H-thieno[3,2-d]pyrimidin-4-one (I-25) was prepared analogously except in step 1, [4-(tert-butoxycarbonylamino)-phenyl]boronic acid was replaced with 4-butoxy-3-chloro-phenylboronic acid (CASRN 480438-55-9) and steps 2 and 3 were omitted.

1-(4-Chloro-benzyl)-6-p-tolyl-1H-thieno[3,2-d]pyrimidin-4-one (I-29) was prepared analogously except in step 1, [4-(tent-butoxycarbonylamino)-phenyl]boronic acid was replaced with 4-tert-butyl-phenylboronic acid (CASRN 123324-71-0), 34 was replaced with 30 and steps 2 and 3 were omitted.

N-{4-[1-(4-Chloro-benzyl)-4-oxo-1,4-dihydro-thieno[3,2-d]pyrimidin-6-yl]-phenyl}-acetamide (I-37) was prepared by acetylation of 38b with acetic anhydride in the presence of pyridine and DMAP.

4-[1-(4-Chloro-benzyl)-4-oxo-1,4-dihydro-thieno[3,2-d]pyrimidin-6-yl]-benzonitrile (I-52) was prepared analogously except in step 1, [4-(tert-butoxycarbonylamino)-phenyl]boronic acid was replaced with 4-cyano-phenylboronic acid (CASRN 126747-14-6) and steps 2 and 3 were omitted.

4-[1-(4-Chloro-benzyl)-4-oxo-1,4-dihydro-thieno[3,2-d]pyrimidin-6-yl]-benzoic acid (I-56) was isolated from the Suzuki coupling with 4-cyano-phenylboronic acid (CASRN 126747-14-6) used to prepare I-52.

1-(4-Chloro-benzyl)-6-(4-pyrrolidin-1-yl-phenyl)-1H-thieno[3,2-d]pyrimidin-4-one (I-56) was prepared analogously except in step 1, [4-(tert-butoxycarbonylamino)-phenyl]boronic acid was replaced 4-(pyrrolidin-1-yl)-benzene boronic acid (CASRN 229009-41-0) and steps 2 and 3 were omitted.

Pyrrolidine-1-carboxylic acid {4-[1-(4-chloro-benzyl)-4-oxo-1,4-dihydro-thieno[3,2-d]pyrimidin-6-yl]-phenyl}-amide (I-54) can be prepared analogously except in step 3 the urea can be prepared by treating 38b sequentially with DCI and pyrrolidine.

### Example 2

### N-{2-Chloro-4-[1-(4-chloro-benzyl)-4-oxo-1,4-dihydro-thieno[3,2-d]pyrimidin-6-yl]-phenyl}-4-hydroxy-butyramide (I-30)

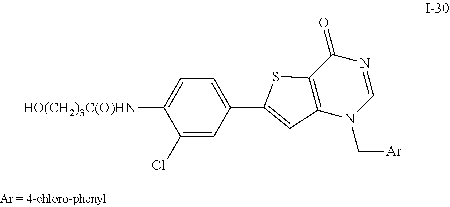

step 1—To a slurry of 34 (500 mg, 0.85 mmol), 4-amino-3-chlorophenylboronic acid pinacol ester (465 mg, 1.84 mmol) and Pd(PPh₃)₄ (250 mg) in DMF (5 mL) was added aq. sat'd. Na₂CO₃ (3.5 mL). The reaction mixture was stirred at 100° C. until the starting material was consumed. The reaction mixture was cooled to RT then quenched H₂O. The resulting precipitate was filtered then sequentially washed with hexanes/EtOAc and hexanes/DCM to yield 417 mg of 6-(4-amino-3-chloro-phenyl)-1-(4-chloro-benzyl)-1H-thieno[3,2-d]pyrimidin-4-one (40): MS calcd for C₁₉H₁₃Cl₂N₃OS [M+H]⁺ 402. Found, 402.

step 2—AlMe₃ (0.25 mL, 2.0 M, 0.5 mmol) was added to a slurry of 40 (50 mg, 0.12 mmol) in DCM (0.5 mL). The mixture was stirred for 15 min then γ-butyrolactone (0.02 mL) was added and the mixture was stirred at 30° C. for 48 h. The mixture was cooled to RT, 1N HCL was added and the solid was collected. This solid was purified on a preparative SiO₂ TLC developed with 30% hexanes/70% Magic to afford 10 mg of N-{2-chloro-4-[1-(4-chloro-benzyl)-4-oxo-1,4-dihydro-thieno[3,2-d]pyrimidin-6-yl]-phenyl}-4-hydroxy-butyramide (I-30): MS calcd for C₂₃H₁₉Cl₂N₃O₃S [M+H]⁺ 488. Found, 488.

¹H NMR (DMSO-d₆, 300 MHz): δ 9.57 (broad s, 1H), 8.67 (s, 1H), 7.99-7.87 (m, 3H), 7.75-7.69 (m, 1H) 7.45 (s, 4H), 5.49 (s, 2H), 4.51 (t, 1H), 3.46 (q, 2H), 2.49 (t, 2H), 1.81-1.68 (m, 2H).

I-35 was prepared analogously except in step 2, γ-butyrolactone was replaced with α-methyl-butyrolactone.

### Example 3

### Pyridine-2-carboxylic acid {2-chloro-4-[1-(4-chloro-benzyl)-4-oxo-1,4-dihydro-thieno[3,2-d]pyrimidin-6-yl]-phenyl}-amide (I-1)

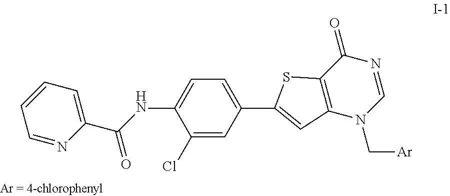

Pyridine-2-carbonyl chloride hydrochloride (40 mg, 0.23 mmol) was added to a slurry of 40 (50 mg, 0.12 mmol) and TEA (0.05 mL, 0.37 mmol) in DCM maintained at 0° C. The reaction mixture was stirred overnight and allowed to warm to RT. The reaction mixture was stirred at 35° C. for 3 d. The solid was collected, washed sequentially with DCM, MeOH, and H₂O to afford 30 mg of I-1: MS calcd. for C₂₅H₁₆Cl₂N₄O₂S [M+H]⁺ 507. Found, 507: ¹H NMR (DMSO-d₆, 300 MHz): δ 10.78 (broad s, 1H), 8.81-8.76 (m, 1H), 8.69 (s, 1H), 8.55 (d, 1H), 8.28-8.06 (m, 3H), 7.99 (s, 1H), 7.90-7.82 (m, 1H), 7.80-7.72 (m, 1H), 7.48 (m, 4H), 5.49 (s, 2H).

### Example 4

### N-{4-[1-(4-Chloro-benzyl)-4-oxo-1,4-dihydro-thieno[3,2-d]pyrimidin-6-yl]-2-fluoro-phenyl}-benzamide (I-14)

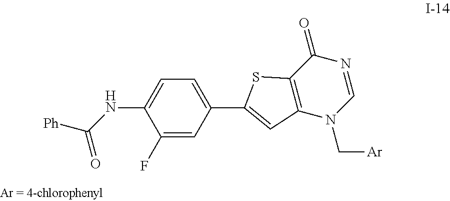

step 1—To a slurry of 34 (200 mg, 0.56 mmol), 4-Boc-amino-3-fluorophenylboronic acid (200 mg, 0.78 mmol) and Pd(PPh₃)₄ (90 mg) in DMF (3 mL) was added sat'd. aq. NaHCO₃ (1.2 mL). The reaction mixture was stirred at 70° C. until all starting material was consumed, cooled to RT and quenched with H₂O. The solution was extracted with EtOAc, dried (Na₂SO₄), filtered and concentrated. The crude product was purified by SiO₂ chromatography eluting with a Magic/DCM gradient (2% to 30% Magic) to afford 200 mg of tent-butyl {4-[1-(4-chloro-benzyl)-4-oxo-1,4-dihydro-thieno[3,2-d]pyrimidin-6-yl]-2-fluoro-phenyl}-carbamate (42): MS calcd for C₂₄H₂₁ClFN₃O₃S [M+H]⁺ 486. Found, 486.

step 2—HCl-dioxane (1 mL, 4.0 M solution) was added to 42 (70 mg). The reaction mixture was stirred at RT until all starting material was consumed. The reaction was concentrated to afford 6-(4-amino-3-fluoro-phenyl)-1-(4-chloro-benzyl)-1H-thieno[3,2-d]pyrimidin-4-one (44) as the HCl salt, which was used without further purification in the next step.

step 3—To a slurry of the HCl salt 44 from step 2 and TEA (33 μL, 0.24 mmol) in DCM maintained at 0° C. was added benzoyl chloride (17 μL, 0.14 mmol). The reaction mixture was stirred overnight and at RT. The mixture was purified on a preparative SiO₂ TLC plate developed with 40% Magic/60% DCM, followed by a SiO₂ column chromatography eluting with a Magic/DCM gradient (0% to 20% Magic) to afford 1.8 mg of I-14: MS calcd for C₂₆H₁₇ClFN₃O₂S [M+H]⁺ 490. Found, 490: ¹H NMR (DMSO-d₆, 300 MHz): δ 10.28 (broad s, 1H), 8.69 (s, 1H), 8.03-7.97 (m, 2H), 7.97 (s, 1H), 7.88-7.78 (m, 2H), 7.69-7.49 (m, 4H), 7.48 (m, 4H), 5.49 (s, 2H).

### Example 5

### 2-Amino-pyrimidine-4-carboxylic acid {4-[1-(4-chloro-benzyl)-4-oxo-1,4-dihydro-thieno[3,2-d]pyrimidin-6-yl]-phenyl}-amide (I-8)

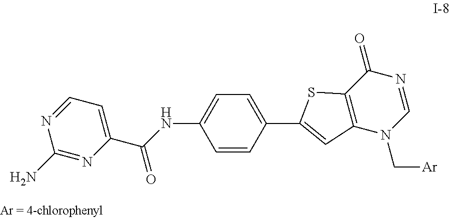

step 1—HCl-dioxane (1 mL, 4.0 M) was added to 38a (70 mg) and the reaction mixture stirred at RT until all starting material was consumed. The solvent was removed in vacuo to afford 38b as an HCl salt which was used in the next step without further purification.

step 2—To a solution of the HCl salt of 38b, 2-amino-pyrimidine-4-carboxylic acid (30 mg, 0.22 mmol) and EEDQ (54 mg, 0.22 mmol) in DMF was added TEA (56 μL, 0.41 mmol). The reaction mixture was heated to 60° C. More reagent was added and the reaction was stirred at 65° C. until all 38b was consumed. The reaction was concentrated in vacuo. The crude product was purified by SiO₂ chromatography eluting with a Magic/DCM gradient (0% to 20% Magic) which resulted in a slightly impure I-8, which was sequentially washed with DCM and MeOH to afford 6.3 mg of the desired product: MS calcd for C₂₄H₁₇ClN₆O₂S [M+H]⁺ 489. Found, 489: ¹H NMR (DMSO-d₆, 300 MHz): δ 10.49 (broad s, 1H), 8.66 (s, 1H), 8.53 (d, 1H), 7.99-7.79 (m, 5H), 7.48 (m, 4H), 7.16 (d, 1H), 6.95 (bs, 2H), 5.49 (s, 2H).

5-Methyl-furan-2-carboxylic acid {4-[1-(4-chloro-benzyl)-4-oxo-1,4-dihydro-thieno[3,2-d]pyrimidin-6-yl]-phenyl}-amide (I-3) was made analogously except 2-amino-pyrimidine-4-carboxylic acid was replaced with 5-methyl-furan-2-carboxylic acid (CASRN 1917-15-3).

2-Amino-N-{4-[1-(4-chloro-benzyl)-4-oxo-1,4-dihydro-thieno[3,2-d]pyrimidin-6-yl]-phenyl}-isonicotinamide (I-9) was made analogously except 2-amino-pyrimidine-4-carboxylic acid was replaced with 2-amino-isonicotinic acid (CASRN 13362-28-2).

Pyrazine-2-carboxylic acid {4-[1-(4-chloro-benzyl)-4-oxo-1,4-dihydro-thieno[3,2-d]pyrimidin-6-yl]-phenyl}-amide (I-12) was made analogously except 2-amino-pyrimidine-4-carboxylic acid was replaced with pyrazine-2-carboxylic acid (CASRN 98-97-5).

5-Methyl-1H-pyrazole-3-carboxylic acid {4-[1-(4-chloro-benzyl)-4-oxo-1,4-dihydro-thieno[3,2-d]pyrimidin-6-yl]-phenyl}-amide (I-15) was made analogously except 2-amino-pyrimidine-4-carboxylic acid was replaced with 5-methyl-1H-pyrazole-3-carboxylic acid (CASRN 402-61-9).

4-Methyl-oxazole-5-carboxylic acid {4-[1-(4-chloro-benzyl)-4-oxo-1,4-dihydro-thieno[3,2-d]pyrimidin-6-yl]-phenyl}-amide (I-18) was made analogously except 2-amino-pyrimidine-4-carboxylic acid was replaced with 4-methyl-5-oxazolecarboxylic acid (CASRN 2510-32-9).

Isoxazole-5-carboxylic acid {4-[1-(4-chloro-benzyl)-4-oxo-1,4-dihydro-thieno[3,2-d]pyrimidin-6-yl]-phenyl}-amide (I-32) was made analogously except 2-amino-pyrimidine-4-carboxylic acid was replaced with 5-isoxazolecarboxylic acid (CASRN 21169-71-1).

### Example 6

### N-{4-[1-(4-Chloro-benzyl)-4-oxo-1,4-dihydro-thieno[3,2-d]pyrimidin-6-yl]-phenyl}-2,5-difluoro-benzamide (I-7)

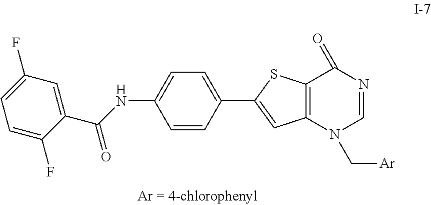

step 1—To a solution of the HCl salt of 38b (50 mg) and TEA (35 μL, 0.25 mmol) in DCM maintained at 0° C. was added 2,5-difluoro-benzoyl chloride (26 mg, 0.15 mmol). The reaction mixture was stirred for 2 h and warmed to RT. The solid was collected and washed with DCM to afford 16 mg of I-7: MS calcd for C₂₆H₁₆ClF₂N₃O₂S [M+H]⁺ 508. Found, 508. ¹H NMR (DMSO-d₆, 300 MHz): δ 10.70 (broad s, 1H), 8.72 (s, 1H), 8.00-7.40 (m, 12H), 5.49 (s, 2H).

### Example 7

### Pyridine-2-carboxylic acid {6-[1-(4-chloro-benzyl)-4-oxo-1,4-dihydro-thieno[3,2-d]pyrimidin-6-yl]-pyridin-3-yl}-amide (I-40)

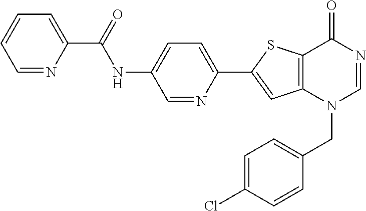

step 1—To a slurry of 34 (150 mg, 0.42 mmol), 5-aminopyridine-2-boronic acid pinacol ester (150 mg, 0.68 mmol; CASRN 1176723-60-6) and Pd(PPh₃)₄ (150 mg) in DMF (5 mL) was added sat'd. aq. Na₂CO₃ (3 mL). The reaction mixture was stirred at 95° C. until all starting material was consumed, cooled to RT, and quenched with H₂O. The solid was filtered, sequentially washed with hexanes/EtOAc and hexanes/DCM to afford 25 mg of 6-(5-amino-pyridin-2-yl)-1-(4-chloro-benzyl)-1H-thieno[3,2-d]pyrimidin-4-one (46): MS calcd for C₁₈H₁₃ClN₄OS [M+H]⁺ 469. Found, 469.

step 2—Pyridine-2-carbonyl chloride hydrochloride (17 mg, 0.10 mmol) was added to a solution of 46 (20 mg, 0.05 mmol) and TEA (17 μL, 0.12 mmol) in DCM maintained at 0° C. The reaction mixture was stirred at 35° C. for 3 h after which additional reagent was added, and the mixture was stirred for an additional 2 h at 35° C. The reaction was concentrated in vacuo. The crude product was purified by SiO₂ chromatography eluting with a Magic/CH₂Cl₂ gradient (0% to 20% Magic) to afford slightly impure I-40, which was washed with H₂O and DCM to give 1 mg of the desired product. MS calcd for C₂₄H₁₆ClN₅O₂S [M+H]⁺ 474. Found, 474; ¹H NMR (DMSO-d₆, 300 MHz): δ 10.99 (broad s, 1H), 9.11 (s, 1H), 8.80-8.73 (m, 1H), 8.61 (s, 1H), 8.53-8.45 (m, 1H), 8.22-8.02 (m, 3H), 7.98 (s, 1H), 7.75-7.66 (m, 1H), 7.48 (s, 4H), 5.49 (s, 2H).

### Example 8

### Pyridine-2-carboxylic acid {5-[1-(4-chloro-benzyl)-4-oxo-1,4-dihydro-thieno[3,2-d]pyrimidin-6-yl]-pyridin-2-yl}-amide (I-41)

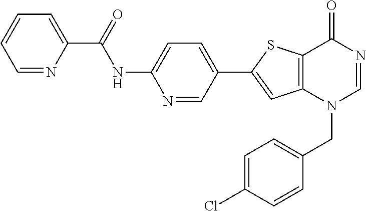

step 1—To a slurry of 34 (100 mg, 0.45 mmol), 2-amino-pyridine-5-boronic acid, pinacol ester (0.100 g, 0.45 mmol, CASRN 827614-64-2) and Pd(PPh₃)₄ (70 mg) in DMF (1.2 mL) was added sat'd. aq. Na₂CO₃ (0.7 mL). The reaction mixture was stirred at 70° C. until all starting material was consumed, allowed to cool to RT, and quenched with H₂O. The solid was collected, washed sequentially with hexanes/EtOAc and hexanes/DCM to afford 70 mg of 6-(6-amino-pyridin-3-yl)-1-(4-chloro-benzyl)-1H-thieno[3,2-d]pyrimidin-4-one (48): MS calcd for C₁₈H₁₃ClN₄OS [M+H]⁺ 369. Found, 369.

step 2—Pyridine-2-carbonyl chloride hydrochloride (52 mg, 0.29 mmol) was added to a solution of 48 (60 mg, 0.16 mmol) and TEA (50 μL, 0.36 mmol) in DCM maintained at 0° C. The reaction mixture was stirred and allowed to warm to RT overnight. Additional reagent was added, and the mixture was stirred for an additional 10 d. The solid was collected, sequentially washed with DCM, MeOH and H₂O to afford 10 mg of I-41. MS calcd for C₂₄H₁₆ClN₅O₂S [M+H]⁺ 474. Found, 474: ¹H NMR (DMSO-d₆, 300 MHz): δ 10.62 (broad s, 1H), 8.89 (d, 1H), 8.80-8.76 (m, 1H), 8.69 (s, 1H), 8.43-8.08 (m, 4H), 7.99 (s, 1H), 7.79-7.71 (m, 1H), 7.52-7.43 (m, 4H), 5.49 (s, 2H).

### Example 9

### 6-(4-Benzyloxy-3-chloro-phenyl)-1-(4-chloro-benzyl)-1H-thieno[3,2-d]pyrimidin-4-one (I-44)

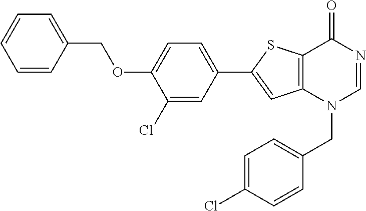

A microwave vial containing a small stir bar was charged with 34 (142.5 mg) and 4-benzyloxy-3-chloro-boronic acid (266 mg, CASRN 845551-44-2), K₂CO₃ (372 mg) and Pd(dppf)Cl₂ (30 mg). Dioxane (4 mL) and H₂O (1 mL) were added, and argon was briefly bubbled through the solution. The vial was capped and irradiated in a microwave synthesizer at 125° C. for 45 min. After cooling the vial was opened and the contents poured into brine and the solution was twice extracted with DCM. The combined extracts were dried (MgSO₄), filtered and concentrated. The crude product was purified by SiO₂ chromatography eluting with stepwise gradient (5% MeOH in 1/1 hexanes/EtOAc, then 5% MeOH in EtOAc) to afford 29 mg of I-44 as a tan solid.

### Example 10

### 1-(4-Chloro-benzyl)-6-[4-(pyridin-2-ylmethoxy)-phenyl]-1H-thieno[3,2-d]pyrimidin-4-one (I-28)

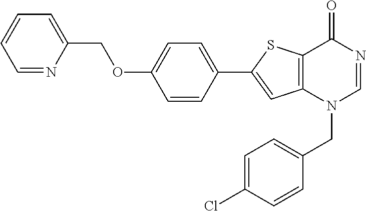

step 1—A flask was charged with 4-(4,4,5,5-tetramethyl-[1,3,2]dioxaborolan-4-yl)-phenol (510 mg, CASRN 269409-70-3, 50) and pyridin-2-yl methanol (280 mg), PPh₃ (690 mg) then dissolved in DCM (20 mL). The solution was cooled to 0° C. and diisopropyl azodicarboxylate (0.57 mL) was added and the cooling bath was removed. After 30 min, the reaction was passed through a SiO₂ pad and solvent was removed. The crude product was purified by SiO₂ chromatography eluting with 20% EtOAc/hexane to afford 610 mg of 2-[4-(4,4,5,5-tetramethyl-[1,3,2]dioxaborolan-2-yl)-phenoxymethyl]-pyridine (52) as a white solid.

step 2—A microwave vial containing a small stir bar was charged with 34 (108 mg) and 52 (135 mg), followed by Na₂CO₃ (255 mg) and Pd(PPh₃)₄ (15 mg). Toluene (2.5 mL), EtOH (1 mL) and H₂O (0.5 mL), then argon was briefly bubbled through the solution. The vial was sealed and irradiated in a microwave synthesizer to 110° C. for 45 min. The reaction mixture was cooled and diluted with DCM and the organic extracts were washed sequentially with H₂O and brine. The extract was dried (MgSO₄), filtered and concentrated. The resulting solid was triturated with EtOAc, MeOH and hexane to afford 91 mg of I-28 as a solid.

1-(4-Chloro-benzyl)-6-[4-(pyridin-3-ylmethoxy)-phenyl]-1H-thieno[3,2-d]pyrimidin-4-one (I-50) was prepared analogously except in step 1, pyridin-4-yl methanol was replaced with pyridin-3-yl methanol. The product obtained by triturating with DCM and hexane exhibited a melting point of 255-257° C.

1-(4-Chloro-benzyl)-6-[4-(pyridin-2-ylmethoxy)-phenyl]-1H-thieno[3,2-d]pyrimidin-4-one (I-58) was prepared analogously except in step 1, pyridin-42-yl methanol was replaced with pyridin-2-yl methanol. The product obtained by triturating with DCM and hexane exhibited a melting point of 257-259° C.

### Example 11

### 6-[4-(4-Amino-6-methyl-pyrimidin-2-ylmethoxy)-phenyl]-1-(4-chloro-benzyl)-1H-thieno[3,2-d]pyrimidin-4-one (I-45)

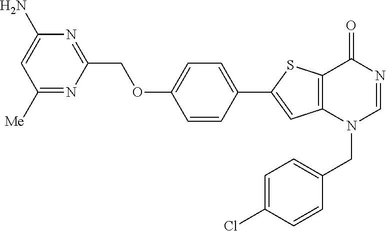

step 1—A solution of the 2-iodomethyl-6-methyl-pyrimidin-4-ylamine (580 mg, CASRN 108260-15-7) and 50 (500 mg) in acetone (40 mL) was treated with K₂CO₃ (1 g) and heated overnight at 50° C. under argon. The reaction was cooled and poured into 1:1 EtOAc/hexane (200 mL). The solution was washed sequentially with H₂O and brine, dried (MgSO₄), filtered and concentrated. The crude product was purified by SiO₂ chromatography eluting with an EtOAc/hexane gradient (50 to 100% EtOAc) to afford 6-methyl-2-[4-(4,4,5,5-tetramethyl-[1,3,2]dioxaborolan-2-yl)-phenoxymethyl]-pyrimidin-4-ylamine (54).

step 2—A microwave vial containing a small stir bar was charged with 34 (103 mg) and 54 (123 mg), Na₂CO₃ (122 mg) and Pd(PPh₃)₄ (10 mg) then DCM (2.5 mL), MeOH (2.5 mL) and H₂O (0.1 mL) were added. Argon was briefly bubbled through the solution, the vial was sealed and irradiated in a microwave synthesizer at 105° C. for 35 min. After the reaction was cooled the mixture was diluted with DCM and washed sequentially with H₂O and brine. The extract was dried (MgSO₄), filtered and evaporated. Triturating of the resulting solid with hot DCM, MeOH and hexanes afforded 74 mg of I-45 as a yellow solid: mp 272-274 C.

The following were prepared analogously by cross-coupling the appropriate boronic acid prepared by alkylation of 50 with the indicated benzyl bromide: 1-(4-chloro-benzyl)-6-[4-(3-trifluoromethyl-phenoxymethyl)-phenyl]-1H-thieno[3,2-d]pyrimidin-4-one (3-trifluoromethyl-benzylbromide, CASRN 402-23-2), 1-(4-chloro-benzyl)-6-[4-(4-chloro-benzyloxy)-phenyl]-1H-thieno[3,2-d]pyrimidin-4-one (4-chloro-benzyl bromide, CASRN 622-95-7), 1-(4-chloro-benzyl)-6-(4-phenoxymethyl-phenyl)-1H-thieno[3,2-d]pyrimidin-4-one (benzyl bromide), 1-(4-chloro-benzyl)-6-[4-(4-chloro-3-methyl-phenoxymethyl)-phenyl]-1H-thieno[3,2-d]pyrimidin-4-one (CASRN 117890-58-1), 1-(4-chloro-benzyl)-6-[4-(4-methoxy-benzyloxy)-phenyl]-1H-thieno[3,2-d]pyrimidin-4-one (I-11, CASRN 2746-25-0), 1-(4-chloro-benzyl)-6-[4-(4-fluoro-benzyloxy)-phenyl]-1H-thieno[3,2-d]pyrimidin-4-one (I-5, CASRN 459-46-1), 1-(4-chloro-benzyl)-6-[4-(2-methoxy-benzyloxy)-phenyl]-1H-thieno[3,2-d]pyrimidin-4-one (1-(bromomethyl)-2-methoxy-benzene, CASRN 52289-93-7), 1-(4-chloro-benzyl)-6-[4-(2-chloro-5-trifluoromethyl-phenoxymethyl)-phenyl]-1H-thieno[3,2-d]pyrimidin-4-one (I-31, 2-chloro-5-(trifluoromethyl)benzyl bromide, CASRN 237761-77-20), 1-(4-chloro-benzyl)-6-[3-chloro-4-(pyridin-2-ylmethoxy)-phenyl]-1H-thieno[3,2-d]pyrimidin-4-one (I-6, 2-iodomethylpyridine, CASRN 929876-97-1), 1-(4-chloro-benzyl)-6-[4-(pyrazin-2-ylmethoxy)-phenyl]-1H-thieno[3,2-d]pyrimidin-4-one (I-16, 2-iodomethyl-pyrazine, CASRN 120276-51-9), 1-(4-chloro-benzyl)-6-[3-(4-fluoro-benzyloxymethyl)-phenyl]-1H-thieno[3,2-d]pyrimidin-4-one (I-34, 1-(bromomethyl)-4-fluoro-benzene, CASRN 459-46-1).

### Example 12

### 1-(4-Chloro-benzyl)-6-[4-(pyridin-2-ylmethylsulfanyl)-phenyl]-1H-thieno[3,2-d]pyrimidin-4-one (I-48)

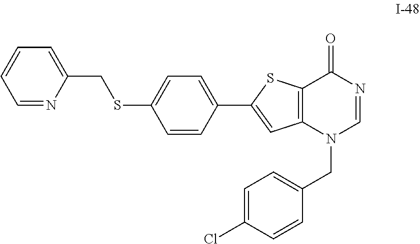

step 1—p-Bromothiophenol (380 mg, 2 mmol) and 2-bromomethylpyridine hydrobromide (500 mg, 2 mmol, CASRN 31106-82-8)) were stirred overnight with Na₂CO₃ (1 g) in DMF (10 mL). The mixture was partitioned between Et₂O and water and the product purified by SiO₂ chromatography eluting with an EtOAc/hexane gradient (0 to 20% EtOAc) to afford 2-(4-bromo-phenylsulfanylmethyl)-pyridine (56).

step 2—A solution of 56, KOAc (800 mg), bis-(pinacolato)diboron (1 g), and PdCl₂(dppf) (150 mg) in dioxane was heated at 100° C. overnight. The mixture was partitioned between Et₂O and H₂O. The borinate ester was purified by SiO₂ chromatography eluting with an EtOAc/hexane gradient (0 to 25% EtOAc) to afford 2-[4-(4,4,5,5-tetramethyl-[1,3,2]dioxaborolan-2-yl)-phenylsulfanylmethyl]-pyridine (58)

step 3—Palladium-catalyzed cross-coupling of 58 and 34 was carried out in accord with the procedure described in step 2 of Example 11.

1-(4-Chloro-benzyl)-6-[4-(pyridin-3-ylmethylsulfanyl)-phenyl]-1H-thieno[3,2-d]pyrimidin-4-one (I-51) was prepared analogously except in step 1, 2-bromomethylpyridine hydrobromide was replaced with 3-bromomethylpyridine hydrobromide (CASRN 4916-55-6).

1-(4-Chloro-benzyl)-6-[4-(pyridin-4-ylmethylsulfanyl)-phenyl]-1H-thieno[3,2-d]pyrimidin-4-one (I-60) was prepared analogously except in step 1, 2-bromomethylpyridine hydrobromide was replaced with 4-bromomethylpyridine hydrobromide (CASRN 73870-24-3).

6-(4-Benzylsulfanyl-phenyl)-1-(4-chloro-benzyl)-1H-thieno[3,2-d]pyrimidin-4-one (I-20) was prepared analogously except in step 1, 2-bromomethylpyridine hydrobromide was replaced with benzyl bromide.

### Example 13

### 6-(4-Benzylamino-phenyl)-1-(4-chloro-benzyl)-1H-thieno[3,2-d]pyrimidin-4-one (I-49)

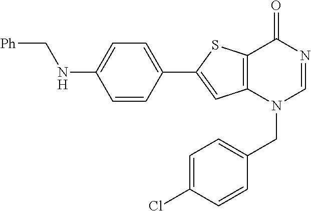

A suspension of 38a DCM (10 mL) and an equal volume of 4M HCl in dioxane. was stirred for 1 h then evaporated. To a solution containing one half of the resulting product in DMF (1 mL) was added benzyl bromide (60 μL, 1 eq.) and DIPEA (200 μL). After the reaction was complete the mixture was partitioned between EtOAc and water. The product was purified on a preparative SiO₂ TLC plate developed with 5% MeOH/DCM) to afford 10 mg of 60.

1-(4-Chloro-benzyl)-6-{4-[(pyridin-2-ylmethyl)-amino]-phenyl}-1H-thieno[3,2-d]pyrimidin-4-one was prepared analogously except in 2-bromomethyl-pyridine was used in place of benzyl bromide.

### Example 14

### 6-[4-(7-Amino-5-methyl-pyrazolo[1,5-a]pyrimidin-2-yl)-phenyl]-1-(4-methyl-benzyl)-1H-thieno[3,2-d]pyrimidin-4-one (I-38)

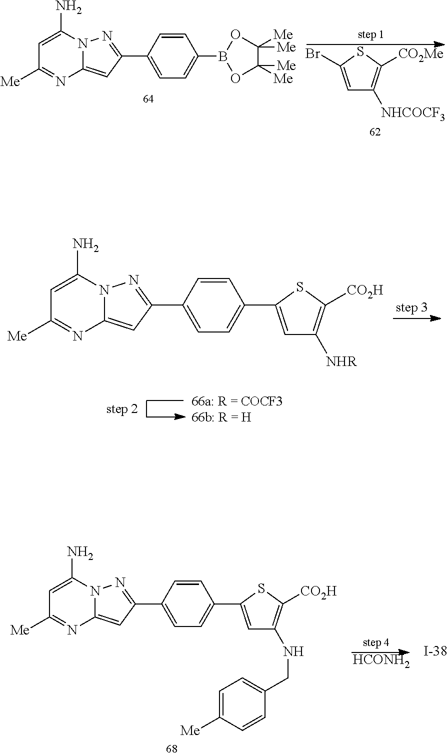

step 1—To a slurry of methyl 5-bromo-3-(2,2,2-trifluoro-acetylamino)-thiophene-2-carboxylate (62, 200 mg, 0.60 mmol) and 5-methyl-2-[4-(4,4,5,5-tetramethyl-[1,3,2]dioxaborolan-2-yl)-phenyl]-pyrazolo[1,5-a]pyrimidin-7-ylamine (64, 316 mg, 0.9 mmol, CASRN 642589-62-0) in DMF (6 mL) and aq. sat'd. Na₂CO₃ (3 mL) was added Pd(PPh₃)₄ (98 mg). The reaction mixture was heated to 100° C. for 3 h and then cooled to RT. The solvent was removed and the crude product purified by SiO₂ chromatography (Isco) eluting with 10% MeOH/DCM to afford 75 mg of 66a: MS calcd for C₂₁H₁₆F₃N₅O₃S [M+H]⁺ 476. Found, 476. (In some cases, the trifluoroacetamide group was (partially) hydrolyzed).

step 2—Aqueous K₂CO₃ was added to a solution of 66a in MeOH. After the reaction was complete, the solvent was removed, and the crude product 66b was used in the next step without further purification.

step 3—To a solution of 66b (75 mg, 0.2 mmol) and 4-methyl-benzaldehyde (67, 25 μL, 0.36 mmol) in DCM (1 mL) and HOAc (21 μL) cooled to 0° C. was added NaBH(OAc)₃ (53 mg, 0.25 mmol). The reaction mixture was stirred and warmed to RT. Additional aldehyde and NaBH(OAc)₃ were added to push the reaction to completion. The mixture was purified on a preparative SiO₂ plate developed with 10% MeOH/DCM to afford 100 mg of 68: MS calcd for C₂₇H₂₅N₅O₂S [M+H]⁺ 484. Found, 484.

step 4 —To a solution of 68 (100 mg, 0.21 mmol) in formamide (0.06 mL) and DMF (1 mL) was added NaOMe (0.15 mL, 25% in MeOH). The reaction mixture was heated to 100° C. then cooled to RT and purified on a preparative SiO₂ TLC plate developed with 30% MeOH/DCM to afford 5 mg of I-38: MS calcd for C₂₇H₂₂N₆OS [M+H]⁺ 479. Found, 479. ¹H NMR (MeOH-d₄, 300 MHz, 2 Hs not observed): δ 8.49 (s, 1H), 8.18-8.09 (m, 2H), 7.85-7.79 (m, 2H), 7.67 (s, 1H), 7.35-7.20 (m, 4H), 6.69 (s, 1H), 6.05 (s, 1H), 5.51 (s, 2H), 2.41 (s, 3H), 2.31 (s, 3H).

### Example 15

### 1-(4-Methyl-benzyl)-6-(4-pyrazolo[1,5-a]pyrimidin-2-yl-phenyl)-1H-thieno[3,2-d]pyrimidin-4-one (I-57)

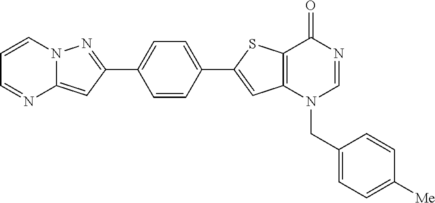

step 1—To a slurry of 62 (100 mg, 0.29 mmol) and 2-[4-(4,4,5,5-tetramethyl-[1,3,2]dioxaborolan-2-yl)-phenyl]-pyrazolo[1,5-a]pyrimidine (141 mg, 0.44 mmol, CASRN 642589-50-6) in DMF (2 mL) and sat'd. aq. Na₂CO₃ (1.3 mL) was added Pd(PPh₃)₄ (47 mg). The reaction mixture was heated to 100° C. for 2 h and then cooled to RT. The solvent was removed and the residue purified by SiO₂ chromatography (Isco) eluting with a Magic/DCM gradient (0% to 20% Magic) to afford 100 mg 3-(4-methyl-benzylamino)-5-(4-pyrazolo[1,5-a]pyrimidin-2-yl-phenyl)-thiophene-2-carboxylic acid methyl ester (70): MS calcd for C₂₆H₂₂N₄O₂S [M+H]⁺ 455. Found, 455.

step 2—To a solution of 70 (100 mg, 0.22 mmol) in formamide (0.06 mL) and DMF (1 mL) was added NaOMe (0.14 mL, 25% in MeOH). The reaction mixture was heated to 120° C. then cooled to RT, and purified on a preparative SiO₂ TLC plate developed with 30% Magic/DCM to afford 2 mg of I-57: MS calcd for C₂₆H₁₉N₅OS [M+H]⁺ 450. Found, 450; ¹H NMR (DMSO-d₆, 300 MHz): δ 9.15 (d, 1H), 8.69 (s, 1H), 8.55 (d, 1H), 8.18 (d, 2H), 7.99 (s, 1H), 7.92 (d, 2H), 7.26 (s, 1H), 7.35 (d, 2H), 7.19 (s, 2H), 7.06 (m, 1H), 5.46 (s, 2H), 2.24 (s, 3H).

6-(4-Furo[3,2-b]pyridin-2-yl-phenyl)-1-(4-methyl-benzyl)-1H-thieno[3,2-d]pyrimidin-4-one (I-19) is prepared analogously except in step 1, 2-[4-(4,4,5,5-tetramethyl-[1,3,2]dioxaborolan-2-yl)-phenyl]-pyrazolo[1,5-a]pyrimidine is replaced with 2-[4-(4,4,5,5-tetramethyl-[1,3,2]dioxaborolan-2-yl)-phenyl]-furo[3,2-b]pyridine

### Example 16

### 1-(4-Chloro-benzyl)-6-[4-(5-methyl-[1,3,4]oxadiazol-2-yl)-phenyl]-1H-thieno[3,2d]pyrimidin-4-one (I-33)

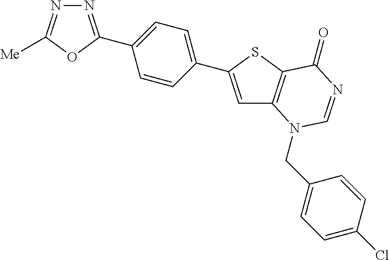

To a slurry of 34 (50 mg, 0.14 mmol) and 2-methyl-5-[4-(4,4,5,5-tetramethyl-[1,3,2]dioxaborolan-2-yl)-phenyl]-[1,3,4]oxadiazole (43 mg, 0.15 mmol, CASRN 913835-70-8) in DMF (1 mL) and sat'd. aq. Na2CO3 (1.4 mL) was added Pd(PPh3)4 (46 mg). The reaction mixture was heated to 100° C. for 1 h then cooled to RT. The solvent was removed and the crude product purified by SiO2 chromatography (Isco) eluting with a Magic/DCM gradient (0% to 30% Magic) to afford 12 mg of I-33: MS calcd for C22H15ClN4O2S [M+H]⁺ 435. Found, 435. ¹H NMR (DMSO-d₆, 300 MHz): 8.70 (s, 1H), 8.12-8.00 (m, 4H), 8.03 (s, 1H), 7.47 (s, 4H), 5.52 (s, 2H), 2.61 (s, 3H).

### Example 17

### 6-[4-(7-Amino-5-methyl-pyrazolo[1,5-a]pyrimidin-2-yl)-3-chloro-phenyl]-1-(4-chloro-benzyl)-1H-thieno[3,2-d]pyrimidin-4-one (I-2)

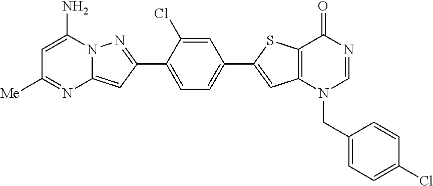

To a slurry of 34 (50 mg, 0.14 mmol) and 4-(7-amino-5-methyl-pyrazolo[1,5-a]pyrimidin-2-yl)-3-chloro-phenyl boronic acid (51 mg, 0.17 mmol) in DMF (1 mL) and sat'd. aq. Na₂CO₃ (0.7 mL) was added Pd(PPh₃)₄ (23 mg). The reaction mixture was heated to 100° C. for 1 h then cooled to RT. The solvent was removed and the crude product purified by SiO₂ chromatography (Isco) eluting with a Magic/DCM gradient (0% to 20% Magic) to afford 16 mg of I-2: MS calcd for C₂₆H₁₈Cl₂N₆OS [M+H]⁺ 533. Found, 533. ¹H NMR (DMSO-d₆, 300 MHz): δ 8.69 (s, 1H), 8.14-8.09 (m, 3H), 7.87 (dd, 1H), 7.62 (broad s, 2H), 7.48 (s, 4H), 6.77 (s, 1H), 6.04 (s, 1H), 5.52 (s, 2H), 2.38 (s, 3H).

6-[4-(7-amino-5-methyl-pyrazolo[1,5-a]pyrimidin-2-yl)-3-chloro-phenyl]-1-(4-methyl-cyclohexylmethyl)-1H-thieno[3,2-d]pyrimidin-4-one was prepared analogously except 34 was replaced with 36 to afford 6 mg of I-4: MS calcd for C₂₇H₂₇ClN₆OS [M+H]⁺ 519. Found, 519; ¹H NMR (DMSO-d₆, 300 MHz): δ 8.42 (s, 1H), 8.20-8.18 (m, 2H), 8.12 (d, 1H), 7.94 (dd, 1H), 7.62 (broad s, 2H), 6.78 (s, 1H), 6.05 (s, 1H), 4.10 (d, 2H), 2.39 (s, 3H), 1.90-0.90 (m, 10H), 0.85 (d, 3H).

6-[6-(7-Amino-5-methyl-pyrazolo[1,5-a]pyrimidin-2-yl)-pyridin-3-yl]-1-(4-methyl-cyclohexylmethyl)-1H-thieno[3,2-d]pyrimidin-4-one was prepared analogously except 34 was replaced with 36 and 4-(7-amino-5-methyl-pyrazolo[1,5-a]pyrimidin-2-yl)-3-chloro-phenyl boronic acid was replaced with 2-(7-amino-5-methyl-pyrazolo[1,5-a]pyrimidin-2-yl)-pyridin-5-ylboronic acid to afford 13 mg of I-43: MS calcd for C₂₆H₂₇N₇OS [M+H]⁺ 486. Found, 486. ¹H NMR (DMSO-d₆, 300 MHz): δ 9.19 (s, 1H), 8.48-8.40 (m, 2H), 8.29 (d, 1H), 8.18 (s, 1H), 7.65 (broad s, 2H), 6.85 (s, 1H), 6.04 (s, 1H), 4.10 (d, 2H), 2.39 (s, 3H), 1.90-0.90 (m, 10H), 0.85 (d, 3H).

6-[6-(7-Amino-5-methyl-pyrazolo[1,5-a]pyrimidin-2-yl)-pyridin-3-yl]-1-(4-chloro-benzyl)-1H-thieno[3,2-d]pyrimidin-4-one was prepared analogously except 4-(7-amino-5-methyl-pyrazolo[1,5-a]pyrimidin-2-yl)-3-chloro-phenyl boronic acid was replaced with 2-(7-amino-5-methyl-pyrazolo[1,5-a]pyrimidin-2-yl)-pyridin-5-yl boronic acid to afford to afford 2 mg of I-42: calcd for C₂₅H₁₈ClN₇OS [M+H]⁺ 500. Found, 500. ¹H NMR (DMSO-d₆, 300 MHz): δ 9.10 (s, 1H), 8.71 (s, 1H), 8.37-8.25 (m, 2H), 8.06 (s, 1H), 7.65 (broad s, 2H), 7.54-7.45 (m, 4H), 6.84 (s, 1H), 6.05 (s, 1H), 5.52 (s, 2H), 2.39 (s, 3H).

### Example 18

### 6-[4-(2-Amino-pyrimidin-4-yl)-phenyl]-1-(4-chloro-benzyl)-1H-thieno[3,2-d]pyrimidin-4-one (I-10)

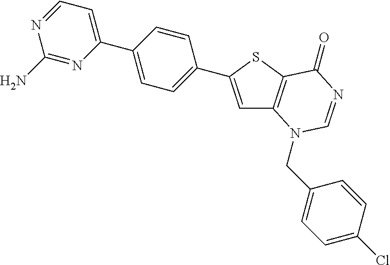

step 1—To a slurry of 4-(4-bromo-phenyl)-pyrimidin-2-ylamine (300 mg, 1 mmol), bis(pinacolato)diborane (400 mg, 1.6 mmol), KOAc (330 mg, 3.3 mmol) in dioxane (7 mL) was added Pd(dppf)Cl₂ (60 mg). The reaction mixture was heated to 100° C. for 2 h then cooled to RT. The solvent was removed. The residue was dissolved in DCM, washed with H₂O, and dried (Na₂SO₄). Upon concentration, some of the product precipitated out and was collected to afford 90 mg of a mixture of 4-[4-(4,4,5,5-tetramethyl-[1,3,2]dioxaborolan-2-yl)-phenyl]-pyrimidin-2-ylamine (70) and the corresponding boronic acid.

step 2—To a slurry of 34 (60 mg, 1.2 mmol) and a mixture 70 and the corresponding boronic acid (50 mg) in DMF (1.2 mL) and sat'd. aq. Na₂CO₃ (0.7 mL) was added d(PPh₃)₄ (50 mg). The reaction mixture was heated to 70 C for 1.5 h then cooled to RT. H₂O and EtOAc were added and the resulting precipitate was collected to afford 12 mg of I-10: MS calcd for C₂₃H₁₆ClN₅OS [M+H]⁺ 446. Found, 446. ¹H NMR (DMSO-d₆, 300 MHz): δ 8.69 (s, 1H), 8.36 (d, 1H), 8.24-8.16 (m, 2H), 7.99 (m, 1H), 7.97-7.90 (m, 2H), 7.48 (s, 4H), 7.19 (d, 1H), 6.71 (broad s, 2H), 5.52 (s, 2H).

### Example 19

### 6-(4-Hydroxymethyl-phenyl)-1-(4-methyl-benzyl)-1H-thieno[3,2-d]pyrimidin-4-one (I-53)

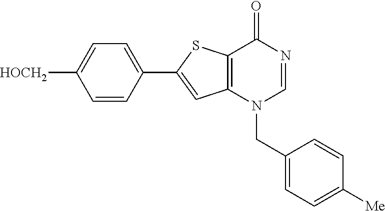

step 1—To a slurry of methyl 5-bromo-3-(4-methyl-benzylamino)-thiophene-2-carboxylate (154 mg, 0.45 mmol) and [4-(4,4,5,5-tetramethyl-[1,3,2]dioxaborolan-2-yl)-phenyl]-methanol (158 mg, 0.68 mmol) in DMF (4.5 mL) and sat'd. aq. Na₂CO₃ (1.9 mL) was added Pd(PPh₃)₄ (73 mg). The reaction mixture was heated to 100° C. for 3 h then cooled to RT. The solvent was removed and the crude product purified by SiO₂ chromatography (Isco) eluting with an EtOAc/hexane gradient (0% to 30% EtOAc) to afford 255 mg of methyl 5-(4-hydroxymethyl-phenyl)-3-(4-methyl-benzylamino)-thiophene-2-carboxylate (72): MS calcd for C₂₁H₂₁NO₃S [M+H]⁺ 368. Found, 368.

step 2—To a solution of 72 (255 mg, 0.7 mmol) in formamide (0.2 mL) and DMF (4 mL) was added NaOMe (0.38 mL, 25% in MeOH). The reaction mixture was heated to 100° C. The reaction was cooled to RT and purified on a preparative SiO₂ TLC plated developed with 30% MeOH/DCM to afford 26 mg of I-53: MS calcd for C₂₁H₁₈N₂O₂S [M+H]⁺ 363. Found, 363; ¹H NMR (MeOH-d₄, 300 MHz, 1H not observed): δ 8.58 (s, 1H), 7.74-7.69 (m, 2H), 7.60 (s, 1H), 7.48-7.41 (m, 2H), 7.30-7.19 (m, 4H), 5.50 (s, 2H), 4.65 (s, 2H), 2.31 (s, 3H).

### Example 20

### HCV NS5B RNA Polymerase Activity

The enzymatic activity of HCV polymerase (NS5B570n-Con1) was measured as the incorporation of radiolabeled nucleotide monophosphates into acid insoluble RNA products. Unincorporated radiolabeled substrate was removed by filtration and scintillant was added to the washed and dried filter plate containing radiolabeled RNA product. The amount of RNA product generated by NS5B570n-Con1 at the end of the reaction was directly proportional to the amount of light emitted by the scintillant.

The HCV polymerase used in the enzymatic activity assay is a 21 amino acid C-terminal deletion of full-length HCV polymerase derived from HCV Con1 strain, genotype 1b (GenBank accession number AJ242654) (NS5B570n-Con1). The NS5B570n-Con1 was sub-cloned downstream to the T7 promoter of the plasmid expression construct pET17b and transformed into E. coli strain BL21(DE3) pLysS for protein expression. A single colony was used to start an innoculum for a 10 L culture in LB media supplemented with 100 μg/mL ampicillin at 37° C. Protein expression was induced by the addition of 0.25 mM isopropyl-β-D-thiogalactopyranoside (IPTG) when the optical density of the culture at 600 nM was 0.8. Induction of protein expression was carried out at 30° C. for 16 h after which the cells were harvested by centrifugation. NS5B570n-Con1 was purified to homogeneity using a three-column purification protocol including subsequent column chromatography on Ni-NTA, SP-Sepharose HP and Superdex 75 resins.

Enzymatic reactions in the presence of cIRES RNA template (see paragraph [00213]) contained 20 nM cIRES RNA, 20 nM NS5B570n-Con1 enzyme, 0.5 μCi of tritiated UTP (Perkin Elmer catalog no. TRK-412; specific activity: 30 to 60 Ci/mmol;), 1 μM each ATP, CTP, and GTP, 40 mM Tris-HCl pH 8.0, 40 mM NaCl, 4 mM DTT (dithiothreitol), 4 mM MgCl₂, 5 μl of compound serial diluted in DMSO, and nuclease-free water to a final reaction volume of 50 μl. Enzymatic reactions in the presence of poly A RNA template (see paragraph [00213]) contained 20 nM Poly A:oligo(rU)16 premixed (see section 0004), 20 nM NS5B570n-Con1 enzyme, 1 μCi of tritiated UTP (Perkin Elmer catalog no. TRK-412; specific activity: 30 to 60 Ci/mmol), 40 mM Tris-HCl pH 8.0, 40 mM NaCl, 4 mM DTT (dithiothreitol), 4 mM MgCl₂, 5 μl of compound serial diluted in DMSO, and nuclease-free water to a final reaction volume of 50 μl. Reaction mixtures were assembled in 96-well filter plates (cat #MADVN0B, Millipore Co.) and incubated for 2 h at 30° C. Reactions were stopped by addition of 10% final (v/v) trichloroacetic acid and incubated for 40 min at 4° C. Reactions were filtered, washed with 8 reaction volumes of 10% (v/v) trichloroacetic acetic acid, 4 reaction volumes of 70% (v/v) ethanol, air dried, and 25 μl of scintillant (Microscint 20, Perkin-Elmer) was added to each reaction well.

Two RNA templates were used to assay compounds described herein. The cIRES RNA template was 377 nucleotide long and consisted of a partial complementary sequence (36 nucleotides) of the core protein, followed by 341 nucleotide of the complementary sequence of the internal ribosome entry site. The poly A RNA template (GE Amersham catalog number 27-4110) was a homopolymeric RNA pre-annealed to a oligo(rU)16 primer at a molar ratio of 3-to-1 (primer-template).

The amount of light emitted from the scintillant was converted to counts per minute (CPM) on a Topcount® plate reader (Perkin-Elmer, Energy Range: Low, Efficiency Mode: Normal, Count Time: 1 min, Background Subtract: none, Cross talk reduction: Off).

Data was analyzed in Excel® (Microsoft®) and ActivityBase® (Idbs®). The reaction in the absence of enzyme was used to determine the background signal, which was subtracted from the enzymatic reactions. Positive control reactions were performed in the absence of compound, from which the background corrected activity was set as 100% polymerase activity. All data was expressed as a percentage of the positive control. The compound concentration at which the enzyme-catalyzed rate of RNA synthesis was reduced by 50% (IC₅₀) was calculated by fitting

$$Y = \% Min + \frac{( \% Max - \% Min )}{[ 1 + \frac{X}{( {IC}_{50} ) S} ]} \qquad ( i )$$

equation (i) to the data where “Y” corresponds to the relative enzyme activity (in %), “% Min” is the residual relative activity at saturating compound concentration, “% Max” is the relative maximum enzymatic activity, “X” corresponds to the compound concentration, and “S” is the Hill coefficient (or slope).

### HCV Replicon Assay

This assay measures the ability of the compounds of formula I to inhibit HCV RNA replication, and therefore their potential utility for the treatment of HCV infections. The assay utilizes a reporter as a simple readout for intracellular HCV replicon RNA level. The Renilla luciferase gene was introduced into the first open reading frame of a genotype 1b replicon construct NK5.1 (N. Krieger et al., J. Virol. 2001 75(10):4614), immediately after the internal ribosome entry site (IRES) sequence, and fused with the neomycin phosphotransferase (NPTII) gene via a self-cleavage peptide 2A from foot and mouth disease virus (M. D. Ryan & J. Drew, EMBO 1994 13(4):928-933). After in vitro transcription the RNA was electroporated into human hepatoma Huh7 cells, and G418-resistant colonies were isolated and expanded. Stably selected cell line 2209-23 contains replicative HCV subgenomic RNA, and the activity of Renilla luciferase expressed by the replicon reflects its RNA level in the cells. The assay was carried out in duplicate plates, one in opaque white and one in transparent, in order to measure the anti-viral activity and cytotoxicity of a chemical compound in parallel ensuring the observed activity is not due to decreased cell proliferation or due to cell death.

HCV replicon cells (2209-23), which express Renilla luciferase reporter, were cultured in Dulbecco's MEM (Invitrogen cat no. 10569-010) with 5% fetal bovine serum (FBS, Invitrogen cat. no. 10082-147) and plated onto a 96-well plate at 5000 cells per well, and incubated overnight. Twenty-four hours later, different dilutions of chemical compounds in the growth medium were added to the cells, which were then further incubated at 37° C. for three days. At the end of the incubation time, the cells in white plates were harvested and luciferase activity was measured by using the R. luciferase Assay system (Promega cat no. E2820). All the reagents described in the following paragraph were included in the manufacturer's kit, and the manufacturer's instructions were followed for preparations of the reagents. The cells were washed once with 100 μl of phosphate buffered saline (pH 7.0) (PBS) per well and lysed with 20 μl of 1×R. luciferase Assay lysis buffer prior to incubation at room temperature for 20 min. The plate was then inserted into the Centro LB 960 microplate luminometer (Berthold Technologies), and 100 μl of R. luciferase Assay buffer was injected into each well and the signal measured using a 2-second delay, 2-second measurement program. IC50, the concentration of the drug required for reducing replicon level by 50% in relation to the untreated cell control value, can be calculated from the plot of percentage reduction of the luciferase activity vs. drug concentration as described above.

WST-1 reagent from Roche Diagnostic (cat no. 1644807) was used for the cytotoxicity assay. Ten microliter of WST-1 reagent was added to each well of the transparent plates including wells that contain media alone as blanks Cells were then incubated for 2 h at 37° C., and the OD value was measured using the MRX Revelation microtiter plate reader (Lab System) at 450 nm (reference filter at 650 nm). Again CC50, the concentration of the drug required for reducing cell proliferation by 50% in relation to the untreated cell control value, can be calculated from the plot of percentage reduction of the WST-1 value vs. drug concentration as described above.

TABLE II

| | HCV Replicon |
|---|---|
| Compound | Activity |
| Number | IC₅₀ (μM) |
| I-38 | 0.0472 |
| I-2 | 0.0041 |

### Example 22

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound according to formula I wherein: 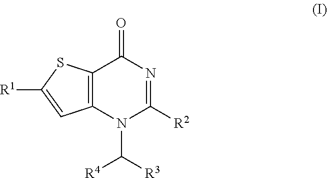
R¹ is phenyl or pyridinyl optionally substituted with one to three groups selected from the group consisting of:
(a) C₁₋₆ alkyl,
(b) C₁₋₆ alkoxy,
(c) halogen,
(d) phenyl-C₁₋₆ alkoxy said phenyl optionally independently substituted by one to three groups selected from C₁₋₃ alkoxy, halogen or C₁₋₃ alkyl or C₁₋₃-haloalkyl,
(e) phenyl,
(f) heteroaryl-C₁₋₃ alkoxy wherein the heteroaryl group is pyridinyl, pyrimidinyl or pyrazinyl said heteroaryl optionally independently substituted by one or two groups selected from amino, C₁₋₆ alkyl, halogen or C₁₋₆ alkoxy;
(g) phenoxymethyl optionally independently substituted by one or two groups selected from amino, C₁₋₆ alkyl, halogen or C₁₋₆ alkoxy;
(h) pyridinylmethylsulfanyl,
(i) heteroaryl wherein the heteroaryl group is pyridinyl, [1,3,4]oxadiazol-2-yl, furo[3,2-b]pyridine-2-yl, pyrazolo[1,5-a]pyrimidin-2-yl and said heteroaryl is optionally independently substituted by one to three groups selected from C₁₋₆ alkyl, C₁₋₆ alkoxy, halogen, amino, C₁₋₃ alkylamino, C₁₋₃ dialkylamino, a cyclic amine,
(j) phenyl-C₁₋₃ alkylsulfanyl,
(k) hydroxy,
(l) halogen,
(m) carboxyl,
(n) cyano,
(p) C₁₋₆ hydroxyalkyl,
(p) CONR^{c}R^{d},
(q) NR^{a}R^{b},
(r) NHC(O)NR^{g}R^{h}, and
(s) hydrogen;
R² is halogen, C₁₋₃ alkyl or C₁₋₃ alkoxy and n is 0 to 2;
R^{a} and R^{b}: (i) taken individually are independently:
(a) C₁₋₆ alkoxycarbonyl,
(b) benzyl,
(c) hydroxy-C₁₋₆ alkanoyl,
(d) C₁₋₆ acyl,
(e) phenylcarbonyl said phenyl optionally independently substituted with one to three groups selected from C₁₋₃ alkoxy, halo or hydroxy,
(f) heteroarylcarbonyl wherein said heteroaryl group is optionally substituted pyrazole, 2-methyl-furan-5-yl-carbonyl, pyrimidinyl-4-carbonyl, oxazol-5-yl-carbonyl, pyrazin-2-yl-carbonyl, pyridinyl-carbonyl said heteroarylcarbonyl optionally substituted by one or two groups independently selected from C₁₋₆ alkyl, C₁₋₆ alkoxy, halogen, amino, C₁₋₃ alkylamino, C₁₋₃ dialkylamino, a cyclic amine or C₁₋₆ hydroxyalkoxy,
(g) hydrogen,
or, (ii) taken together with the nitrogen to which they are attached are a cyclic amine;
R^{c} and R^{d} are independently hydrogen, C₁₋₆ alkyl, phenyl;
R³ is phenyl optionally substituted with one to three groups selected from the group consisting of (a) C₁₋₆ alkyl, (b) C₁₋₆ alkoxy, (c) halogen, (d) NR^{e}R^{f}, (e) cyano, (f) C₁₋₃ haloalkyl and (g) hydroxy, or, C₃₋₇ cycloalkyl optionally with one to three groups selected from C₁₋₄ alkyl, halogen or C₁₋₄ alkoxy;
R^{e} and R^{f} are independently hydrogen, C₁₋₆ alkyl, C₁₋₆ sulfonyl;
R^{g} and R^{h} are independently hydrogen or C₁₋₃ alkyl or together with the nitrogen to which they are attached form a pyrrolidine or a piperidine;
R⁴ is hydrogen or C₁₋₆ alkyl; or,
a pharmaceutically acceptable salt thereof.

2. The compound according to claim 1 comprising a compound of formula Ia wherein: 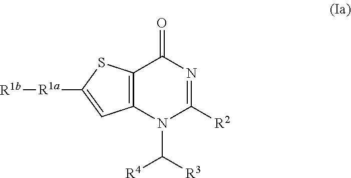
R^{1a} is optionally substituted p-phenylene, R^{1b} is NR^{a}R^{b}, R^{a} is hydrogen and R^{b} is hydroxy-C₁₋₆ alkanoyl, C₁₋₆ acyl, optionally substituted phenylcarbonyl or optionally substituted heteroarylcarbonyl.

3. The compound according to claim 2 wherein R³ is phenyl optionally substituted by halogen or C₁₋₆ alkyl, R^{1a} is p-phenylene optionally further substituted by halogen and R⁴ and R² are hydrogen.

4. The compound according to claim 3 wherein R^{b} is optionally substituted phenylcarbonyl or optionally substituted heteroarylcarbonyl.

5. The compound according to claim 1 comprising a compound of formula Ia wherein R^{1a} is optionally substituted p-phenylene, R^{1b} is optionally substituted heteroaryl and R² and R⁴ are hydrogen.

6. The compound according to claim 5 where R^{1b} is optionally substituted pyrazolo[1,5-a]pyrimidin-2-yl and R³ is phenyl optionally substituted by halogen or C₁₋₆ alkyl.

7. The compound according to claim 6 wherein R^{1b} is 7-amino-5-methyl-pyrazolo[1,5-a]pyrimidin-2-yl.

8. The compound according to claim 1 comprising a compound of formula Ia wherein R^{1a} is optionally substituted p-phenylene and R^{1b} is optionally substituted phenyl-C₁₋₃ alkoxy or optionally substituted heteroaryl-methoxy.

9. The compound according to claim 8 wherein R^{1b} is optionally substituted benzyloxy and R² and R⁴ are hydrogen gen.

10. The compound according to claim 1 said compound selected from the group consisting of:
pyridine-2-carboxylic acid {2-chloro-4-[1-(4-chloro-benzyl)-4-oxo-1,4-dihydro-thieno[3,2-d]pyrimidin-6-yl]-phenyl}-amide;
6-[4-(7-amino-5-methyl-pyrazolo[1,5-a]pyrimidin-2-yl)-3-chloro-phenyl]-1-(4-chloro-benzyl)-1H-thieno[3,2-d]pyrimidin-4-one;
5-methyl-furan-2-carboxylic acid {4-[1-(4-chloro-benzyl)-4-oxo-1,4-dihydro-thieno[3,2-d]pyrimidin-6-yl]-phenyl}-amide;
6-[4-(7-amino-5-methyl-pyrazolo[1,5-a]pyrimidin-2-yl)-3-chloro-phenyl]-1-(4-methyl-cyclohexylmethyl)-1H-thieno[3,2-d]pyrimidin-4-one;
1-(4-chloro-benzyl)-6-[4-(4-fluoro-benzyloxy)-phenyl]-1H-thieno[3,2-d]pyrimidin-4-one;
1-(4-chloro-benzyl)-6-[3-chloro-4-(pyridin-2-ylmethoxy)-phenyl]-1H-thieno[3,2-d]pyrimidin-4-one;
N-{4-[1-(4-chloro-benzyl)-4-oxo-1,4-dihydro-thieno[3,2-d]pyrimidin-6-yl]-phenyl}-2,5-difluoro-benzamide;
2-amino-pyrimidine-4-carboxylic acid {4-[1-(4-chloro-benzyl)-4-oxo-1,4-dihydro-thieno[3,2-d]pyrimidin-6-yl]-phenyl}-amide;
2-amino-N-{4-[1-(4-chloro-benzyl)-4-oxo-1,4-dihydro-thieno[3,2-d]pyrimidin-6-yl]-phenyl}-isonicotinamide;
6-[4-(2-amino-pyrimidin-4-yl)-phenyl]-1-(4-chloro-benzyl)-1H-thieno[3,2-d]pyrimidin-4-one;
1-(4-chloro-benzyl)-6-[4-(4-methoxy-benzyloxy)-phenyl]-1H-thieno[3,2-d]pyrimidin-4-one;
Pyrazine-2-carboxylic acid {4-[1-(4-chloro-benzyl)-4-oxo-1,4-dihydro-thieno[3,2-d]pyrimidin-6-yl]-phenyl}-amide;
6-Biphenyl-4-yl-1-(4-methyl-benzyl)-1H-thieno[3,2-d]pyrimidin-4-one;
N-{4-[1-(4-chloro-benzyl)-4-oxo-1,4-dihydro-thieno[3,2-d]pyrimidin-6-yl]-2-fluoro-phenyl}-benzamide;
5-methyl-1H-pyrazole-3-carboxylic acid {4-[1-(4-chloro-benzyl)-4-oxo-1,4-dihydro-thieno[3,2-d]pyrimidin-6-yl]-phenyl}-amide;
1-(4-chloro-benzyl)-6-[4-(pyrazin-2-ylmethoxy)-phenyl]-1H-thieno[3,2-d]pyrimidin-4-one;
1,5-Dimethyl-1H-pyrazole-3-carboxylic acid {4-[1-(4-chloro-benzyl)-4-oxo-1,4-dihydro-thieno[3,2-d]pyrimidin-6-yl]-phenyl}-amide;
4-methyl-oxazole-5-carboxylic acid {4-[1-(4-chloro-benzyl)-4-oxo-1,4-dihydro-thieno[3,2-d]pyrimidin-6-yl]-phenyl}-amide;
6-(4-Furo [3,2-b]pyridin-2-yl-phenyl)-1-(4-methyl-benzyl)-1H-thieno[3,2-d]pyrimidin-4-one;
6-(4-Benzylsulfanyl-phenyl)-1-(4-chloro-benzyl)-1H-thieno[3,2-d]pyrimidin-4-one;
1-(2-Fluoro-4-methyl-benzyl)-6-phenyl-1H-thieno[3,2-d]pyrimidin-4-one;
6-(4-chloro-phenyl)-1-(4-methyl-benzyl)-1H-thieno[3,2-d]pyrimidin-4-one;
1-(4-chloro-benzyl)-6-{4-[(pyridin-2-ylmethyl)-amino]-phenyl}-1H-thieno[3,2-d]pyrimidin-4-one;
1-(4-chloro-benzyl)-6-(4-phenoxy-phenyl)-1H-thieno[3,2-d]pyrimidin-4-one;
1-(4-chloro-benzyl)-6-(3-chloro-4-propoxy-phenyl)-1H-thieno[3,2-d]pyrimidin-4-one;
N-{2-chloro-4-[1-(4-chloro-benzyl)-4-oxo-1,4-dihydro-thieno[3,2-d]pyrimidin-6-yl]-phenyl}-3-methoxy-benzamide;
1-(2-hydroxy-4-methyl-benzyl)-6-phenyl-1H-thieno[3,2-d]pyrimidin-4-one;
1-(4-chloro-benzyl)-6-[4-(pyridin-2-ylmethoxy)-phenyl]-1H-thieno[3,2-d]pyrimidin-4-one
6-(4-tent-butyl-phenyl)-1-(4-methyl-benzyl)-1H-thieno[3,2-d]pyrimidin-4-one;
N-{2-chloro-4-[1-(4-chloro-benzyl)-4-oxo-1,4-dihydro-thieno[3,2-d]pyrimidin-6-yl]-phenyl}-4-hydroxy-butyramide;
1-(4-chloro-benzyl)-6-[4-(2-chloro-5-trifluoromethyl-phenoxymethyl)-phenyl]-1H-thieno[3,2-d]pyrimidin-4-one;
isoxazole-5-carboxylic acid {4-[1-(4-chloro-benzyl)-4-oxo-1,4-dihydro-thieno[3,2-d]pyrimidin-6-yl]-phenyl}-amide;
1-(4-chloro-benzyl)-6-[4-(5-methyl-[1,3,4]oxadiazol-2-yl)-phenyl]-1H-thieno[3,2-d]pyrimidin-4-one;
1-(4-chloro-benzyl)-6-[3-(4-fluoro-benzyloxymethyl)-phenyl]-1H-thieno[3,2-d]pyrimidin-4-one;
N-{2-chloro-4-[1-(4-chloro-benzyl)-4-oxo-1,4-dihydro-thieno[3,2-d]pyrimidin-6-yl]-phenyl}-4-hydroxy-2-methyl-butyramide;
4-[1-(4-methyl-benzyl)-4-oxo-1,4-dihydro-thieno[3,2-d]pyrimidin-6-yl]-benzoic acid;
N-{4-[1-(4-chloro-benzyl)-4-oxo-1,4-dihydro-thieno[3,2-d]pyrimidin-6-yl]-phenyl}-acetamide;
6-[4-(7-amino-5-methyl-pyrazolo[1,5-a]pyrimidin-2-yl)-phenyl]-1-(4-methyl-benzyl)-1H-thieno[3,2-d]pyrimidin-4-one;
pyridine-2-carboxylic acid {4-[1-(4-chloro-benzyl)-4-oxo-1,4-dihydro-thieno[3,2-d]pyrimidin-6-yl]-phenyl}-amide;
pyridine-2-carboxylic acid {6-[1-(4-chloro-benzyl)-4-oxo-1,4-dihydro-thieno[3,2-d]pyrimidin-6-yl]-pyridin-3-yl}-amide;
pyridine-2-carboxylic acid {5-[1-(4-chloro-benzyl)-4-oxo-1,4-dihydro-thieno[3,2-d]pyrimidin-6-yl]-pyridin-2-yl}-amide;
6-[6-(7-amino-5-methyl-pyrazolo[1,5-a]pyrimidin-2-yl)-pyridin-3-yl]-1-(4-chloro-benzyl)-1H-thieno[3,2-d]pyrimidin-4-one;
6-[6-(7-amino-5-methyl-pyrazolo[1,5-a]pyrimidin-2-yl)-pyridin-3-yl]-1-(4-methyl-cyclohexylmethyl)-1H-thieno[3,2-d]pyrimidin-4-one;
6-(4-benzyloxy-3-chloro-phenyl)-1-(4-chloro-benzyl)-1H-thieno[3,2-d]pyrimidin-4-one;
6-[4-(4-amino-6-methyl-pyrimidin-2-ylmethoxy)-phenyl]-1-(4-chloro-benzyl)-1H-thieno[3,2-d]pyrimidin-4-one;
5-hydroxy-pyridine-2-carboxylic acid {4-[1-(4-chloro-benzyl)-4-oxo-1,4-dihydro-thieno[3,2-d]pyrimidin-6-yl]-phenyl}-amide;
4-[1-(4-chloro-benzyl)-4-oxo-1,4-dihydro-thieno[3,2-d]pyrimidin-6-yl]-N-phenyl-benzamide;
1-(4-chloro-benzyl)-6-[4-(pyridin-2-ylmethylsulfanyl)-phenyl]-1H-thieno[3,2-d]pyrimidin-4-one;
6-(4-benzylamino-phenyl)-1-(4-chloro-benzyl)-1H-thieno[3,2-d]pyrimidin-4-one;
1-(4-chloro-benzyl)-6-[4-(pyridin-3-ylmethoxy)-phenyl]-1H-thieno[3,2-d]pyrimidin-4-one;
1-(4-chloro-benzyl)-6-[4-(pyridin-3-ylmethylsulfanyl)-phenyl]-1H-thieno[3,2-d]pyrimidin-4-one;
4-[1-(4-chloro-benzyl)-4-oxo-1,4-dihydro-thieno[3,2-d]pyrimidin-6-yl]-benzonitrile;
6-(4-hydroxymethyl-phenyl)-1-(4-methyl-benzyl)-1H-thieno[3,2-d]pyrimidin-4-one;
pyrrolidine-1-carboxylic acid {2-chloro-4-[1-(4-chloro-benzyl)-4-oxo-1,4-dihydro-thieno[3,2-d]pyrimidin-6-yl]-phenyl}-amide;
N-{4-[1-(4-chloro-benzyl)-4-oxo-1,4-dihydro-thieno[3,2-d]pyrimidin-6-yl]-2-fluoro-phenyl}-isobutyramide;
1-(4-chloro-benzyl)-6-(4-pyrrolidin-1-yl-phenyl)-1H-thieno[3,2-d]pyrimidin-4-one;
1-(4-methyl-benzyl)-6-(4-pyrazolo[1,5-a]pyrimidin-2-yl-phenyl)-1H-thieno[3,2-d]pyrimidin-4-one;
1-(4-chloro-benzyl)-6-[4-(pyridin-4-ylmethoxy)-phenyl]-1H-thieno[3,2-d]pyrimidin-4-one; and,
1-(4-chloro-benzyl)-6-[4-(pyridin-4-ylmethylsulfanyl)-phenyl]-1H-thieno[3,2-d]pyrimidin-4-one; or,
a pharmaceutically acceptable salt thereof.

11. A method for treating a Hepatitis C Virus (HCV) infection comprising administering to a patient in need thereof, a therapeutically effective quantity of a compound according to claim 1.

12. The method of claim 11 further co-comprising administering at least one immune system modulator and/or at least one antiviral agent that inhibits replication of HCV.

13. The method of claim 12 wherein the immune system modulator is an interferon, interleukin, tumor necrosis factor or colony stimulating factor.

14. The method of claim 13 wherein the immune system modulator is an interferon or chemically derivatized interferon.

15. The method of claim 12 wherein the antiviral compound is selected from the group consisting of a HCV protease inhibitor, another HCV polymerase inhibitor, a HCV helicase inhibitor, a HCV primase inhibitor and a HCV fusion inhibitor.

16. A method for inhibiting replication of HCV in a cell be delivering a compound according to claim 1.

17. A composition comprising a compound according to claim 1 admixed with at least one pharmaceutically acceptable carrier, diluent or excipient.
